(19) United States
(12) Patent Application Publication
(10) Pub. No.: US 2008/0234276 A1
(43) Pub. Date: Sep. 25, 2008

(54) Heterocyclic Triazines as Hypoxic Selective Protein Kinase Inhibitors

(75) Inventors: Robert George Boyle, Cambridge (GB); Stuart Travers, Shefford (GB)

Correspondence Address: HESLIN ROTHENBERG FARLEY & MESITI PC, 5 COLUMBIA CIRCLE, ALBANY, NY, 12203, US

(73) Assignee: SENTINEL ONCOLOGY LIMITED, Bedfordshire (UK)

(21) Appl. No.: 11/815,159

(22) PCT Filed: Feb. 1, 2006
(86) PCT No.: PCT/IB06/01981

§ 371 Date: Apr. 21, 2008

Related U.S. Application Data
(60) Provisional application No. 60/678,202, filed on May 6, 2005.

(30) Foreign Application Priority Data
Feb. 1, 2005 (GB) 0501999.7

(51) Int. Cl. A61K 31/53; C07D 253/10; C12N 9/99; A61P 35/00

(52) U.S. Cl. 514/243; 544/183; 435/184

(57) ABSTRACT

The invention relates to novel heterocyclic triazines which are useful as hypoxic selective cytotoxic agents that mediate and/or inhibit cell proliferation, for example, through the activity of protein kinases. The invention is further related to pharmaceutical compositions containing such compounds and compositions, and to methods of treating cancer as well as other disease states associated with unwanted angiogenesis and/or cellular proliferation by administering effective amounts of such compounds.

This application claims priority under 35 U.S.C §§119 and 120 to U.S. Provisional Patent Application No. 60/678202, filed on May 6, 2005, and to UK Patent Application No. 0501999.7, filed Feb. 1, 2005.

### FIELD OF THE INVENTION

The present invention relates to 3-Substituted 1,2,4-Benzotriazine compounds useful for inhibiting protein kinases, and oxides thereof useful as hypoxia selective prodrugs and radiosensitisers for the treatment of cancer alone or in combination with radiation and/or other anticancer agents.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). They do this by effecting phosphoryl transfer from a nucleoside triphosphate to a target protein that is involved in a signaling pathway. A number of these protein kinases and pathways are stimulated by extracellular stimuli, for which examples include environmental and chemical stress signals (e.g. heat shock, ultraviolet radiation, H₂O₂, osmotic shock), cytokines (e.g. interleukin-1 (IL-1) and Tumour necrosis factor α (TNF-α). Such a signal may effect several pathways within the cell and be important in the progression of many disease states. It is common that many protein kinases are unregulated or constitutively active in cancer. In addition, the cell cycle of all cells is regulated mainly by protein kinases and interference with these can cause cell death by apoptosis or programmed cell death. Protein kinases, where the up regulation leads to inappropriate proliferation include EGFR, ERBB2, PDGFR, cMet, TIE2, RET, FGFR, VEGFR, IGF-1R. Protein kinases involved in signal transduction include PKC, Akt, P70S6, PKA, PDK1, PDK2. Protein kinases involved in cell cycle progression include Cdk1, Cdk2, Cdk4, Myt1, Chk1, Wee1, AuroraA or B, Plk, Bulb1 or 3. Furthermore, protein kinases involved in response to DNA damage, include Chk1, Chk2, ATM, ATR, DNA-PK.+abl, Arg and CKII

Mechanisms of cell proliferation are under active investigation at cellular and molecular levels. At the cellular level, de-regulation of signaling pathways, loss of cell cycle controls, unbridled angiogenesis or stimulation of inflammatory pathways are under scrutiny, while at the molecular level, these processes are modulated by various proteins, among which protein kinases are prominent suspects. Overall abatement of proliferation may also result from programmed cell death, or apoptosis, which is also regulated via multiple pathways, some involving proteolytic enzyme proteins.

Among the candidate regulatory proteins, protein kinases are a family of enzymes that catalyze phosphorylation of the hydroxyl group of specific tyrosine, serine, or threonine residues in proteins. Typically, such phosphorylation dramatically perturbs the function of the protein, and thus protein kinases are pivotal in the regulation of a wide variety of cellular processes, including metabolism, cell proliferation, cell differentiation, and cell survival. Of the many different cellular functions in which the activity of protein kinases is known to be required, some processes represent attractive targets for therapeutic intervention for certain disease states. Two examples are cell-cycle control and angiogenesis, in which protein kinases play a pivotal role; these processes are essential for the growth of solid tumors as well as for other diseases.

Solid tumours, which make up more than 90% of all human cancers, typically have areas of very low oxygenation, or hypoxia (Brown, Molecular Medicine Today, 2000 (vol 6), 157-161). This is because the cells grow faster than the blood supply can keep up with, especially as blood flow is sluggish with very tortuous vessels, and so cells become further away from blood vessels than the diffusion distance of oxygen (100-150 μm). These hypoxic cells are resistant to killing by ionising radiation (Movsas et al., Cancer, 2000, 89, 2018; Rudat et al., Radiother. Oncol., 2000, 57, 31). Hypoxic cells are also considered to compromise response of solid tumours to cytotoxic chemotherapy (Brown and Giaccia, Cancer Res., 1998, 58, 1408). Hypoxic cancer cells also promote malignant progression and make the tumours more likely to metastasize. Typically, the more hypoxic the tumour, the harder it is to cure, a fact that has been demonstrated in many clinical trials. However, hypoxia in tumours can also be exploited and drugs have been developed to take advantage of the different chemical environment within hypoxic cancer cells. One such compound is 3-amino-1,2,4-benzotriazine 1,4-dioxide, named Tirapazamine (TPZ—Denny and Wilson, Exp Opin. Invest. Drugs, 2000, 9, 2889). Although TPZ is showing promising indications of clinical activity, at therapeutic concentrations it also displays considerable toxicity in non-hypoxic cells giving use to unwanted side effects such as nausea, vomiting, diarrhea, neutropenia, thrombocytopenia and muscle cramping. Given these toxic limitations, TPZ cannot be given at doses sufficient enough to fully exploit tumour hypoxia. There is thus a need for compounds that alone, or in combination with TPZ, exhibit enhanced hypoxic specific cytotoxicity.

It has been shown that the mono-N-oxide SR4317 can synergise with both ionising radiation and Tirapazamine by a mechanism that is proposed to be donation of the Oxygen from the mono-N-Oxide, yielding the parent heterocycle (Siim B G. Cancer Research, 2004, 64:736-742).

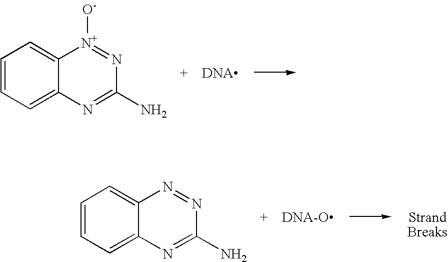

A key parameter for the successful bioreduction under hypoxia is the one-electron reduction potential, E(1). If the E(1) value is too high, reduction will not be limited to hypoxic conditions, and the compound may be toxic to normal cells. Conversely, if the E(1) value is too low, the rate of reduction may be too slow to provide therapeutic benefit. Consequently, the optimal range for hypoxic selective bioreduction appears to be between about −450 mV and −510 mV. Values higher than −300 mV have been found to induce aerobic toxicity, and values lower than −510 mV reduce slowly (Hay M P. J. Med. Chem., 2003, 46:169-182). It has been reported that mono-N-oxides of substituted 3-amino-1,2,4-benzotriazine 1,4-dioxides have E(1) values in the range required for hypoxic bioreduction, and that these values change in line with the substitution patterns (Anderson R F. Org. Biomol. Chem., 2005, 3:2167-2174).

It is an object of the present invention to provide compounds that satisfy this need for enhanced hypoxic specific toxicity. In particular, it is an object of the invention to provide N-oxides capable of selective reduction in a hypoxic tumor environment to become potent protein kinase inhibitors. Further, it is an object to provide compounds that inflict oxidative damage to DNA during their reduction to increase tumor toxicity when administered alone, and/or which potentiate the damage to tumor DNA caused by radiation treatment or drugs such as TPZ when used in combination therewith. It is expected that such compounds of the invention will have little or no protein kinase activity until selectively reduced in the hypoxic environment of a tumor. Such a mechanism will provide a safer protein kinase inhibitor and, in addition, significantly potentiate the initial DNA damaging effect of TPZ when administered together.

Accordingly, it is an object of the present invention to provide a range of heterocyclic N-oxides that are void of kinase activity in their oxidized state, but have E(1) values in the range of −300 mV to −550 mV, preferably −400 mV to −510 mV, and more preferably −450 mV to −510 mV, such that they are selectively reduced under tumor hypoxia to release an active kinase inhibitor. In a second aspect of this invention, it is expected that, when administered in combination with ionising radiation or Tirapazamine or a similar DNA damaging chemotherapeutic (e.g. bleomycin) in a hypoxic environment, these molecules will potentiate the effect of the radiation or chemotherapeutic by: (a) providing an oxygen source to ‘fix’ or make permanent the DNA damage and (b) release an active kinase inhibitor, that will enhance the overall cell killing effect.

It is a farther object of the present invention to provide a range of compounds that inhibit the activity of protein kinases, and which can be readily oxidized to form hypoxia selective prodrugs.

### SUMMARY OF THE INVENTION

The present invention is directed to compounds and methods for treating cancer indications through kinase inhibition and/or DNA oxidative damage. The compounds of the invention are kinase inhibitors, or prodrug compounds which undergo selective reduction in hypoxic tumor environments to form potent inhibitors of kinases, such as abl, Arg, Aurora, CDKs, VEGF (KDR), and CHK-1, or cyclin complexes thereof. As used herein, “hypoxia selective reduction”, “selective reduction in hypoxic tumor environments”, and the like means that, at therapeutic concentrations, reduction of the compound occurs at a level that is therapeutically significant in a hypoxic environment, but therapeutically insignificant in a normoxic environment. Further, in connection with their hypoxia induced reduction, the prodrug compounds of the invention possess, in select situations, the potential to impart oxidative damage to surrounding DNA. This additional functionality may alone provide tumor toxicity, or it may provide synergistic potentiation of the cytotoxic effect of other therapeutic treatments, such as ionizing radiation or chemotherapeutic agents such as TPZ, in hypoxic tumor cells. Accordingly, in one embodiment, the invention is directed to a method of selectively modulating or inhibiting the activity of protein kinases in hypoxic tumor cells. In another embodiment, the invention is directed to a method of selectively causing or potentiating oxidative damage to DNA in hypoxic tumor cells.

In another embodiment, the present invention is directed to certain heterocyclic triazine compounds of the Formula I, which are useful in inhibiting the activity of protein kinases when administered to a mammal:

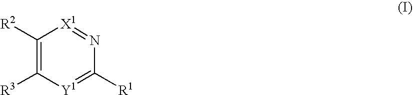

### Wherein:

both X¹ and Y¹ are N-oxide; or

one of X¹ and Y¹ is N and the other is N or N-oxide;

R¹ is H, OH, NH₂, NHR⁴, a 5- to 7-membered heterocyclic ring which is unsaturated and which may contain one or more heteroatoms selected from O, N and S and which is unsubstituted or substituted on any ring carbon or ring heteroatom, or an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system;

R² and R³ are each independently selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸), —N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸)

wherein A is a C₁-C₆ alkylidine group that is optionally interrupted by —O—, —S—, —C(O)— or —N(R⁶), and wherein

R² and R³ may form, together with the carbon atoms to which they are attached, a fused benzene ring or a fused 5- to 7-membered heterocyclic ring which is saturated or unsaturated and which may contain one or more heteroatoms selected from O, N and S, the benzene ring or heterocyclic ring being unsubstituted or substituted;

wherein R⁶ is H, C₁-C₆ alkyl which is unsubstituted or substituted, C₃-C₁₀ cycloalkyl which is unsubstituted or substituted or a 5- to 7-membered heterocyclic ring which is unsaturated or saturated and which contains one or more heteroatoms selected from O, N and S, and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different; and wherein

R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;

p is 0 or an integer from 1 to 5;

q is an integer from 1 to 6;

R⁴ is H, C₁-C₆ alkyl which is unsubstituted or substituted, C₃-C₁₀ cycloalkyl which is unsubstituted or substituted, aryl which is unsubstituted or substituted, a 5- to 7-membered heterocyclic group which is unsaturated or saturated, which contains one or more heteroatoms selected from O, N and S and which is unsubstituted or substituted on any ring carbon or ring heteroatom, the carbocyclic group or heterocyclic group R⁴ being optionally substituted by one or more substituent selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸)—N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, —A-N(R⁶)₂ or -A-N(R⁷)(R⁸) wherein A is a C₁-C₆ alkylidine group that is optionally interrupted by —O—, —S—, —C(O)— or —N(R⁶)—, wherein R⁶, R⁷ and R⁸ are as defined above; and

R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different.

In particularly preferred embodiments, the triazine compounds of Formula I are mono-N oxide or di-N oxide prodrug compounds having one electron reduction potential (E(1)) values less than about −300 mV, and preferably in the range of about −400 mV to about −510 mV, more preferably −450 mV to −510 mV, which are useful as hypoxic selective prodrugs for cytotoxic metabolites that mediate and/or inhibit cell proliferation; for example, through the activity of protein kinases. The preferred compounds of the invention will undergo selective reduction in vivo, under hypoxic conditions, to produce the corresponding mono-N oxide or N-heterocycle active metabolite, which mediates or inhibits kinase activity.

An important requirement for binding to protein kinases is the geometry of the active molecules. The adenine moiety of ATP binds to the kinase active site by hydrogen bonding to a series of backbone amides, a feature that is known as hinge binding and that is a common and important feature of many protein kinase inhibitors (Williams, D. H. Current opinion in Pharmacology, 2002, 2, 567-573. Accordingly, in a preferred embodiment, the compounds of the invention bind to the kinase active site via a comparable hinge binding motif. For example, in one preferred embodiment, once reduced, the azo group previously bearing an oxide moiety bonds to the protein kinase such that it forms part of the hinge binding moiety between the inhibitor and the protein kinase, which renders the kinase incapable of interacting with its natural substrate.

Further, in an additional embodiment of the invention, the oxidizing radical liberated during reduction of the heterocyclic N-oxide prodrug may impart, or potentiate, oxidative damage to the DNA of the tumor cells. Accordingly, this invention further relates to heterocyclic N-oxides having a one electron reduction potential too low to independently cause oxidative damage to tumor DNA in a hypoxic environment, e.g., lower than −510 mV, but that can potentiate the cytotoxic effects of Tirapazamine (TPZ) and/or ionizing radiation, as well as provide active metabolites that have protein kinase inhibitory or modulating effect.

In another preferred embodiment, the invention is directed to compounds of Formula I, wherein one of X¹ and Y¹ is N and the other is N or N-oxide, which inhibit the activity of protein kinases upon exposure thereto.

This invention further relates to pharmaceutical compositions containing compounds of the present invention, and to methods of treating cancer as well as other disease states associated with unwanted angiogenesis and/or cellular proliferation, by administering effective amounts of such compounds.

### DETAILED DESCRIPTION OF PREFERRED EMBODIMENTS OF THE INVENTION

To achieve the afore-mentioned objectives, and in accordance with the purpose of the invention, as embodied and broadly described, one aspect of the invention provides heterocyclic triazines of the Formula I, which are useful in inhibiting the activity of protein kinases when administered to a mammal:

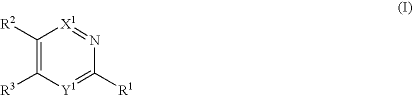

### Wherein:

both X¹ and Y¹ are N-oxide; or

one of X¹ and Y¹ is N and the other is N or N-oxide;

R¹ is H, OH, NH₂, NHR⁴, a 5- to 7-membered heterocyclic ring which is unsaturated and which may contain one or more heteroatoms selected from O, N and S and which is unsubstituted or substituted on any ring carbon or ring heteroatom, or an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system;

R² and R³ are each independently selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶ )CON(R⁷)(R⁸), —N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸)

wherein A is a C₁-C₆ alkylidine group that is optionally interrupted by —O—, —S—, —C(O)— or —N(R⁶), and wherein

R² and R³ may form, together with the carbon atoms to which they are attached, a fused benzene ring or a fused 5- to 7-membered heterocyclic ring which is saturated or unsaturated and which may contain one or more heteroatoms selected from O, N and S, the benzene ring or heterocyclic ring being unsubstituted or substituted;

wherein R⁶ is H, C₁-C₆ alkyl which is unsubstituted or substituted, C₃-C₁₀ cycloalkyl which is unsubstituted or substituted or a 5- to 7-membered heterocyclic ring which is unsaturated or saturated and which contains one or more heteroatoms selected from O, N and S, and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different; and wherein

R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;

p is 0 or an integer from 1 to 5;

q is an integer from 1 to 6;

R⁴ is H, C₁-C₆ alkyl which is unsubstituted or substituted, C₃-C₁₀ cycloalkyl which is unsubstituted or substituted, aryl which is unsubstituted or substituted, a 5- to 7-membered heterocyclic group which is unsaturated or saturated, which contains one or more heteroatoms selected from O, N and S and which is unsubstituted or substituted on any ring carbon or ring heteroatom, the carbocyclic group or heterocyclic group R⁴ being optionally substituted by one or more substituent selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸)—N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(⁷)(R⁸) wherein A is a C₁-C₆ alkylidine group that is optionally interrupted by —O—, —S—, —C(O)— or —N(R⁶)—, wherein R⁶, R⁷ and R⁸ are as defined above; and

R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different.

In particularly preferred embodiments, the compounds of Formula I have one electron reduction potential (E(1)) values less than about −300 mV, and preferably in the range of about −400 mV to about −510 mV, more preferably −450 mV to −510 mV, and are useful as hypoxic selective prodrugs for cytotoxic metabolites that mediate and/or inhibit cell proliferation; for example, through the activity of protein kinases. The preferred compounds of the invention will undergo selective reduction in vivo, under hypoxic conditions, to produce the corresponding mono-N-oxide or the parent N-heterocycle active metabolite, which mediates or inhibits kinase activity.

The compounds of this application have affinity for the following kinases: Arg, Abl, Aurora A, CDK1/cyclinB, CDK2/cyclinE, CHK1, c-RAF, cSRC, EGFR, ErbB4, GFR1, JNK1α1, KDR, MAPK2, MEK1, p70S6K, PDGFRβ, PKC θ, and Plk3, Flt-1, C=Kit, FGFR1, ERBBZ, CMet, TIEZ, RET, VEGFR, IGF-1R, Akt, PKA, P13K, PDK1, PDK2, Cdk2, Cdk4, Ck2, Myt1, Wee1, Auroa B, Plk, Bulb1, Bulb3, Chk2, ATM, ATR, and DNA-PK. In particular, the compounds of this invention are useful for inhibiting: CK2, Arg, Abl, Aurora-A, CDK1/cyclinB, CDK2/cyclinE, CHK1, KDR and p70S6K.

Further, in an additional embodiment of the invention, the oxidising radical liberated during reduction of the heterocyclic N-oxide prodrug may impart, or potentiate, oxidative damage to the DNA of the tumor cells. Accordingly, this invention further relates to heterocyclic N-monoxides having a one electron reduction potential too low to independently cause oxidative damage to tumor DNA in a hypoxic environment, e.g., lower than −510 mV, but that can potentiate the cytotoxic effects of Tirapazamine (TPZ) and/or ionizing radiation, as well as provide active metabolites that have protein kinase inhibitory or modulating effect. The cytotoxic effect of damage to the tumor DNA is further amplified by the release of the kinase inhibitor, when the kinase that is inhibited is involved, directly or indirectly, in the response of the cell to the DNA damage. This effect can be particularly significant when the kinases inhibited are selected from Arg, abl, ATM, Atr, Chk1, Chk2, DNA-PK, and CK11.

In one preferred embodiment, the heterocyclic triazine compound is of the formula (Ia):

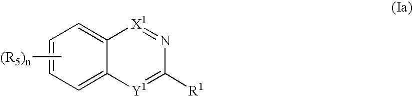

wherein

both X¹ and Y¹ are N-oxide; or

one of X¹ and Y¹ is N and the other is N or N-oxide;

R¹ is as defined above;

each R⁵, which are the same or different, are as defined above for R² and R³; and

n is an integer from 1 to 4.

In another aspect of the invention, di-oxide forms of the heterocyclic triazine compounds herein described are used, such that these compounds provide ‘protected’ protein kinase inhibitors that are released upon being reduced under hypoxic conditions by a process similar to that described for Tirapazamine. The result is masked protein kinase activity until released by a hypoxic environment in the tumour. For example, as shown below, when a di-oxide prodrug of the invention A is administered, under hypoxia, the mono-oxide B and/or the fully reduced parent heterocycle C are produced. While the di-oxide A is void of Protein kinase activity, the mono-oxide B and parent heterocycle C may inhibit Protein kinase activity and potentiate the damage caused by the reductive process of A, or of other DNA damaging therapeutics.

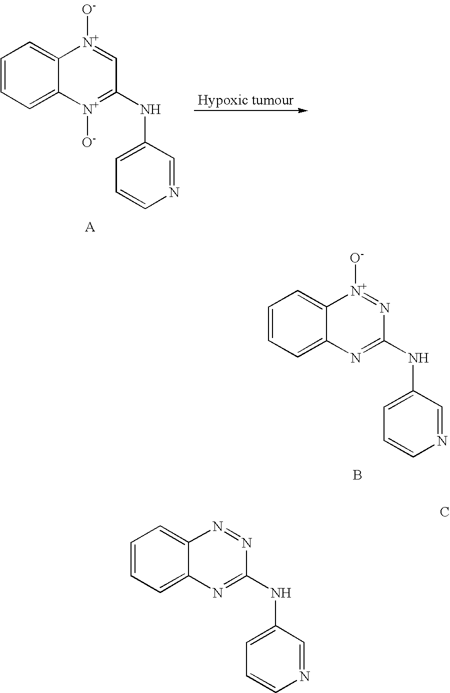

Accordingly, a further aspect of the present invention includes mono-N-oxide and parent heterocycle compounds of the Formula (I) which inhibit the activity of protein kinases upon exposure thereto. The invention thus also provides a method of modulating and/or inhibiting kinase activity by exposing a kinase to a compound of the Formula I or I(a) wherein one of X¹ and Y¹ is N and the other is N or N-oxide.

In the above general formulae a C₁-C₆ alkyl group may be linear or branched. A C₁-C₆ alkyl group is typically a C₁-C₄ alkyl group, for example a methyl, ethyl, n-propyl, i-propyl, n-butyl, sec-butyl or tert-butyl group. A C₁-C₆ alkyl group may be unsubstituted or substituted, typically by one or more of the groups specified above as options for R⁵. More typically, a C₁-C₆ alkyl group is unsubstituted, or substituted by one or more groups selected from halogen, hydroxyl, C₁-C₆ alkoxy, nitro, amino, cyano, aryl which is unsubstituted or substituted, a 5- to 7-membered heterocyclic group as defined above (such as morpholinyl, piperidinyl, piperazinyl or pyridyl), —N(R⁶)₂, —SR⁶ and —COOR⁶ wherein R⁶ is as defined above.

A C₁-C₆ alkyl group substituted by halogen may be denoted by the term “halo-C₁-C₆ alkyl”, which means an alkyl group in which one or more hydrogens is replaced by halo. A halo-C₁-C₆ alkyl group preferably contains one, two or three halo groups. A preferred example of such a group is trifluoromethyl.

A halogen is F, Cl, Br or I. Preferably it is F, Cl or Br.

A C₁-C₆ alkoxy group may be linear or branched. It is typically a C₁-C₄ alkoxy group, for example a methoxy, ethoxy, propoxy, i-propoxy, n-propoxy, n-butoxy, sec-butoxy or tert-butoxy group. A C₁-C₆ alkoxy group may be unsubstituted or substituted, typically by one or more groups selected from those specified above as substituents for C₁-C₆ alkyl.

A C₃-C₁₀ cycloalkyl group may be, for instance, a C₃-C₈ cycloalkyl such as cyclopropyl, cyclobutyl, cyclopentyl, cyclohexyl or cycloheptyl. Typically it is C₃-C₆ cycloalkyl. A C₃-C₁₀ cycloalkyl group may be unsubstituted or substituted, typically by one or more groups selected from those specified above as substituents for C₁-C₆ alkyl.

A C₃-C₁₀ cycloalkoxy group is a group —O-cycloalkyl wherein the cycloalkyl moiety contains from 3 to 10 carbon atoms. Typically it is a C₃-C₈ or C₃-C₆ cycloalkoxy group. It may be, for instance, a cyclopropoxy, cyclobutoxy, cyclopentoxy, cyclohexoxy, cycloheptoxy or cyclooctoxy group.

An alkylidine group is a polymethylene group, i.e. —(CH₂)ₙ— wherein n is a positive integer. Preferably, n is an integer from 1 to 6.

When R² and R³ form, together with the carbon atoms to which they are attached, a benzene ring or a 5- or 6-membered heterocyclic ring, the resulting fused bicyclic heterocycle is typically a benzotriazine, quinazoline, benzopyridazine, tetrahydrobenzotriazine, tetrahydroquinazoline, tetrahydrobenzopyridazine, pyranotriazine, dihydropyranotriazine, pyridotriazine, pyridopyrimidine, pyridopyridazine, pyrimidotriazine, pyrimidopyrimidine, pyrimidopyridazine, pyrrolotriazine, pyrrolopyrimidine, pyrrolopyridazine, oxazolotriazine, oxazoloquinoline, oxazolopyridazine, thienotriazine, thienoquinoline, thienopyridazine, furotriazine, furoquinoline, furopyridazine, thiazolotriazine, thiazoloquinoline, thiazolopyridazine, imadazotriazone, imidazoquinoline or imidazopyridazine.

A thienotriazine may be a thieno[2,3-e]triazine or a thieno[3,2-e]triazine. A pyrrolotriazine may be a pyrrolo[2,3-e]triazine or a pyrrolo[3,2-e]triazine. A furotriazine may be a furo[2,3-e]triazine or a furo[3,2-e]triazine. A thiazolotriazine may be a thiazolo[4,5-e]triazine or a thiazolo[5,4-e]triazine. An oxazolotriazine may be an oxazolo[4,5-e]triazine or an oxazolo[5,4-e]triazine. An imidazotriazine is typically 5H-imidazo[4,5-e]triazine or 7H-imidazo[4,5-e]triazine

A 3- to 9-, or 5- to 7-membered N-containing heterocyclic ring which is unsaturated or saturated and contains 0, 1, or 2 additional heteroatoms selected from O, N, and S may be, for example, imidazolyl, imidazolinoyl, imidazolidinyl, perhydropyridazyl, pyridazyl, pyridyl, pyrrolyl, pyrrolinyl, pyrrolidinyl, pyrazolyl, pyrazinyl, piperidinyl, pyrazolinyl, piperazinyl, pyrimidinyl, pyridazinyl, morpholinyl, thiamorpholinyl, triazolyl, tetrazolyl, isothiazolyl, thiazolyl, thiadiazolyl, thiazolidinyl, oxazolyl, isoxazolyl, oxadiazolyl and oxadiazolidinyl. Preferred examples of such heterocycles are pyridyl, pyrrolyl, pyrrolinyl, piperidinyl, piperazinyl and morpholinyl. The N-containing heterocycle is unsubstituted or substituted on any ring carbon or ring heteroatom, for instance by one or more groups specified above as substituents for C₁-C₆ alkyl. Preferably the substituent is one of the options defined above for R⁵.

A 5- to 7-membered heterocyclic group containing one or more heteroatoms selected from O, N and S is unsaturated or saturated. Suitable examples include those specified above as examples of a 5- to 7-membered N-containing heterocyclic ring. Further examples include furyl, thienyl, pyranyl, tetrahydropyranyl, tetrahydrofuranyl, thiazolyl and thiophenyl rings. Preferably the group is one of the above mentioned N-containing heterocyclic groups or pyrrolyl, furyl, pyridyl, piperidinyl or morpholinyl. The 5- to 7-membered heterocyclic group may be unsubstituted or substituted on any ring carbon atom or ring heteroatom, for instance by one or more of the groups specified above as substituents for C₁-C₆ alkyl. Typically the substituent is halogen, C₁-C₆ alkyl or halo-C₁-C₆ alkyl.

An Aryl group is a carbocyclic aromatic radical containing from 6-14 carbon atoms, preferably 6-10 atoms. Examples include phenyl, napthyl, indenyl and indanyl groups. An aryl group may be unsubstituted or substituted, for instance by one or more of the groups specified above as substituents for C₁-C₆ alkyl. Preferably the substituent is one of the options specified above for R⁵. Typically an aryl group is substituted by C₁-C₆ alkyl, halo-C₁-C₆ alkyl or halogen.

In the structures of formulae (I) and (Ia):

When R², R³ or R⁵ is —SO2N(R⁷)(R⁸), —N(R⁷)(R⁸), —OCH₂(CH₂)ₚN(R⁷)(R⁸), —CH₂(CH₂)ₚN(R⁷)(R⁸), —C(O)NHCH₂(CH₂)ₚN(R⁷)(R⁸), —C(O)N(R⁷)(R⁸), —NH(CH₂)ₚN(R⁷)(R⁸), —N(R⁶)CON(R⁷)(R⁸), —NHC(O)CH₂(CH₂)ₚN(R⁷)(R⁸), or -A-N(R⁷)(R⁸) the groups R⁷ and R⁸ form an N-containing heterocyclic ring as defined above, preferably a morpholino, piperidinyl or piperazinyl group, which is unsubstituted or substituted by C₁-C₆ alkyl. Preferred examples of such heterocyclic options for R⁷ and R⁸ include piperazin-1-yl, 4-methyl-piperazin-1-yl and morpholin-4-yl groups.

When R², R³ or R⁵ is —N(R⁶)₂, OCH₂(CH₂)ₚN(R⁶)₂, —CH₂(CH₂)ₚN(R⁶)₂, —CONHCH₂(CH₂)ₚN(R⁶)₂, CON(R⁶)₂, NH(CH₂)ₚN(R⁶)₂, —NHC(O)CH₂(CH₂)ₚN(R⁶)₂, —N(R⁶)CON(R⁶)₂ or -A-N(R⁶)₂, each R⁶ is typically, independently, H or C₁-C₆ alkyl.

The groups R⁵ in formula I(a) occupy any of the positions 5, 6, 7 and 8 of the benzotriazine ring system. Thus, when n is 1, the benzotriazine is monosubstituted at the 5-, 6-, 7- or 8-position by any of the groups specified above as options for R⁵. When n is 2 the benzotriazine is 5,6-, 5,7-, 6,7-, 6,8- or 7,8-disubstituted by any of the groups specified above as options for R⁵. When n is 3 the benzotriazine is 5,6,7-, 6,7,8- or 5,6,8-trisubstituted by any of the groups specified above as options for R⁵. When n is 4 the benzotriazine is 5,6,7,8-tertasubstituted by any of the groups specified above as options for R⁵. When n is 2 and both R⁵ substituents are ortho to each other and they form, together with the atoms to which they are attached, a fused benzene ring or a fused 5- to 7-membered heterocyclic ring which is saturated or unsaturated and which contains one or more heteroatoms selected from O, N and S, the benzene ring or heterocyclic ring may be substituted with the groups R⁵ as described above or unsubstituted.

There is also provided, in accordance with the invention, a pharmaceutical composition comprising the protein kinase inhibitors of the invention. Preferably, such compositions comprise a hypoxic selective prodrug of the formula I, or more preferably formula I(a), which is converted into an active metabolite exhibiting inhibition of protein kinase activity when reduced in a hypoxic environment. Preferably, the mono-N-oxide moiety of the prodrug has a one electron reduction potential less than about −300 mV, more preferably in the range from about −400 mV to about −510 mV. In addition to compounds of the formula I and I(a), a pharmaceutically acceptable salt or prodrug of such compounds may also be used. Further, as is common in the art, the compounds of the invention, including the salt or prodrug forms, may be formulated into pharmaceutical preparations with conventional carriers, diluents, fillers, surfactants, and excipients known in the art.

There is further provided, in accordance with the invention, a method of using the prodrug compounds to selectively release a protein kinase modulating agent for treating a disease or disorder mediated by inhibition of kinase activity, comprising administering to a patient in need thereof, therapeutically effective amounts of a compound of Formula I or I(a), or a pharmaceutically acceptable salt or prodrug thereof. The method is particularly suitable for treating malignancies or cancer as well as other disease states associated with unwanted angiogenesis and/or cellular proliferation. Thus, the invention is also directed to methods of treating such diseases by administering an effective amount of the inventive agent.

Another aspect of the invention is the use of the heterocyclic triazine-N-oxides herein described having a one electron potential lower than about −300 mV, preferably in the range of from about −400 mV to about −510 mV as radiosentizers or potentiators of oxidative DNA damage caused by chemotherapeutic agents such as Tirapazamine and/or ionizing radiation. When administered concurrently with Tirapazamine and/or ionizing radiation, the selective reduction of these N-oxide prodrugs that occurs in the hypoxic environment of the tumor cells, not only releases protein kinase inhibitors, the liberated oxygen atoms have the potential to increase the effectiveness of the TPZ and/or radiation.

In accordance with these aspects of the invention, a pharmaceutical composition containing a compound of the Formula I or I(a), or a pharmaceutically acceptable salt or prodrug of a compound of the Formula I or I(a) may be used in treating diseases mediated by kinase activity, such as cancer, as well as other disease states associated with unwanted angiogenesis and/or cellular proliferation. Thus, the invention is also directed to methods of treating such diseases by administering an effective amount of the inventive agent.

It is to be understood that both the foregoing general description and the following detailed description of specific compounds of the invention are exemplary and explanatory, and are not intended to limit the invention as claimed. Other objects and features of the invention will become apparent from the practice of the invention and the following detailed description. All references cited in this specification are expressly incorporated herein by reference.

Exemplary compounds of Formula I and I(a) include the mono-N-oxides and parent heterocycles:

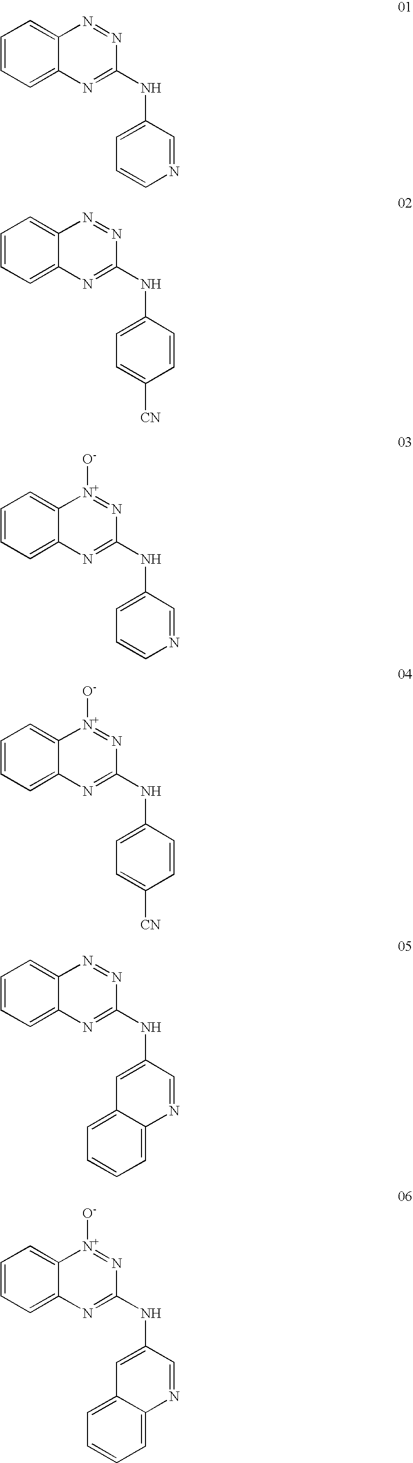

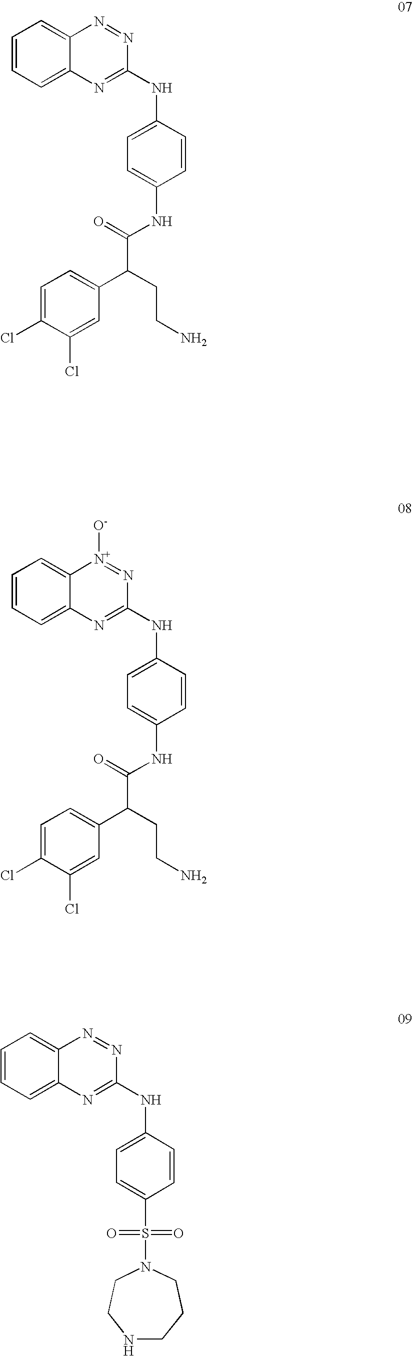

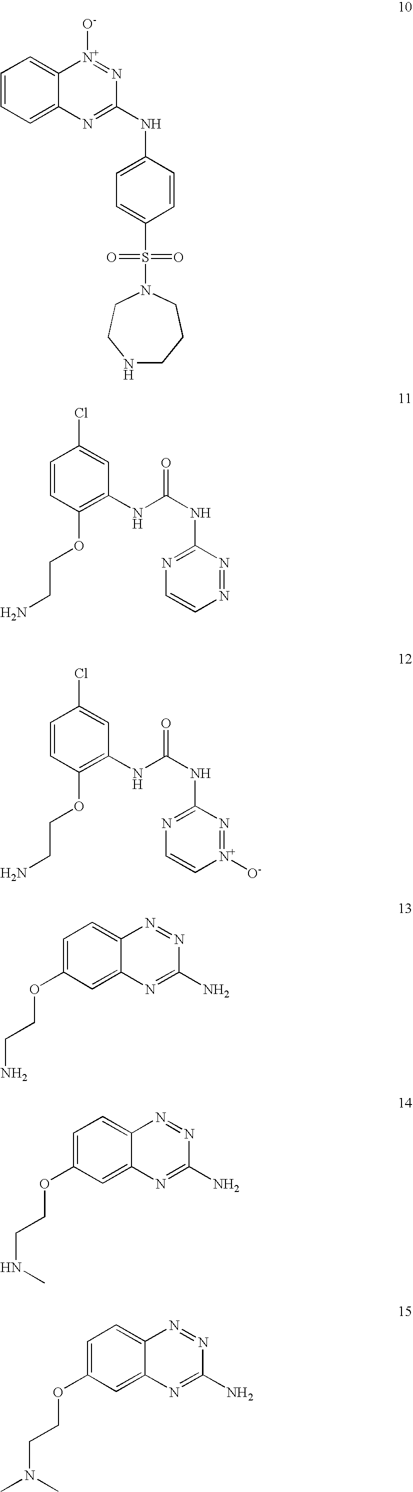

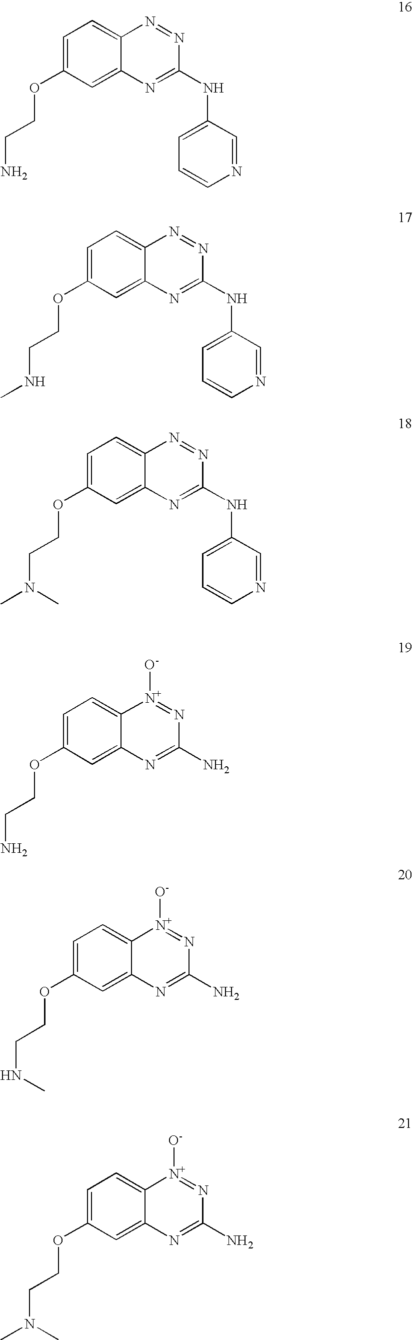

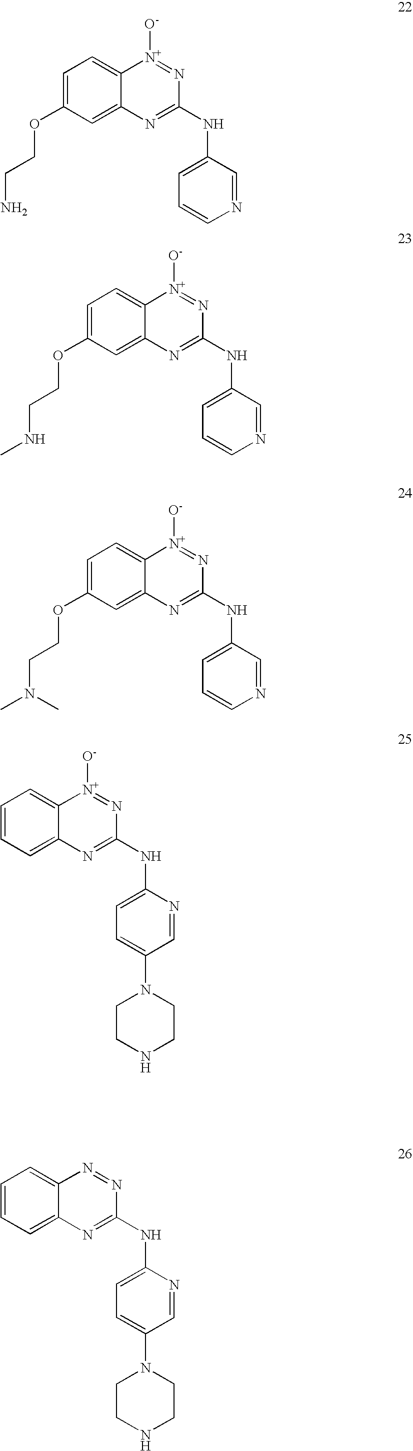

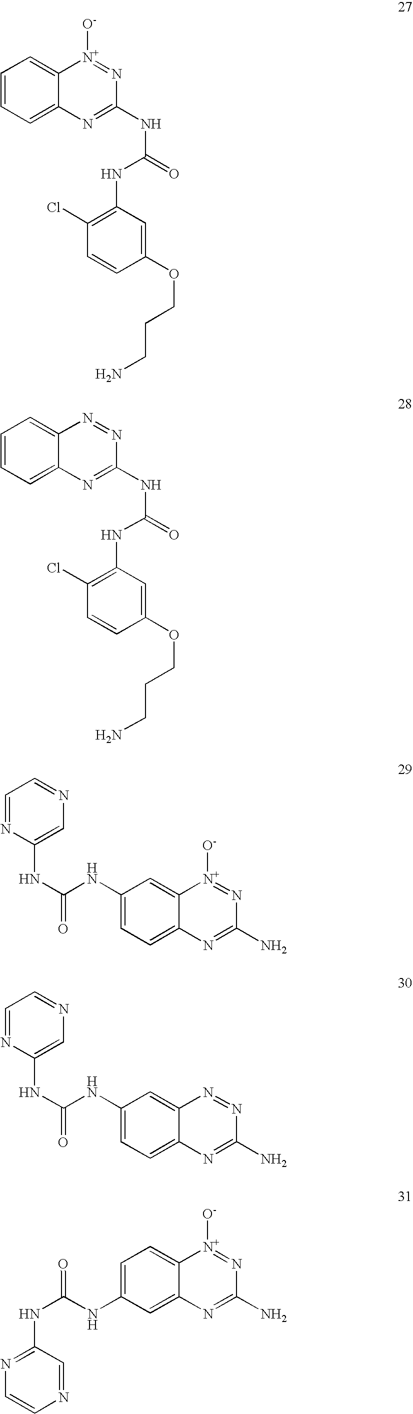

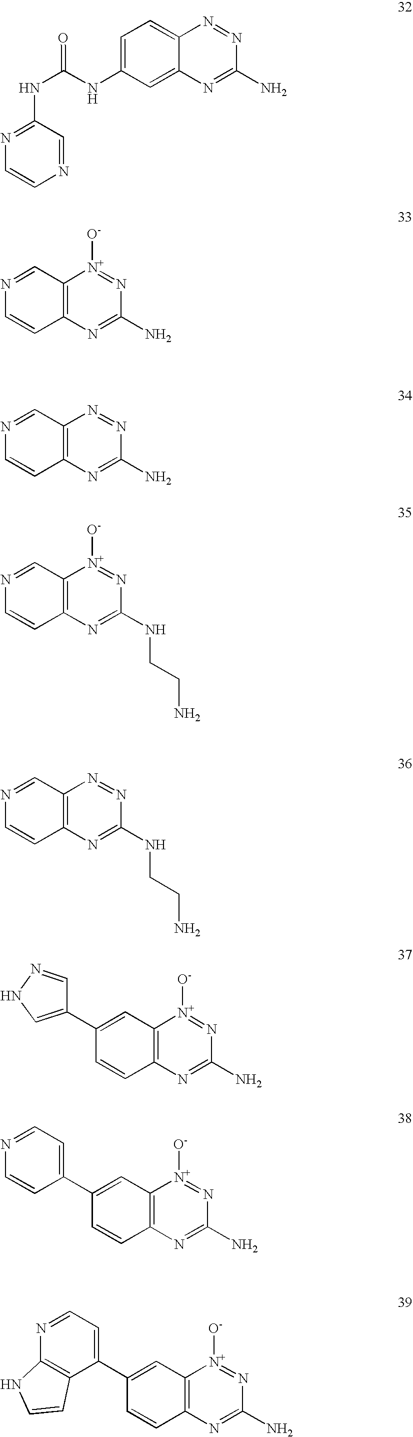

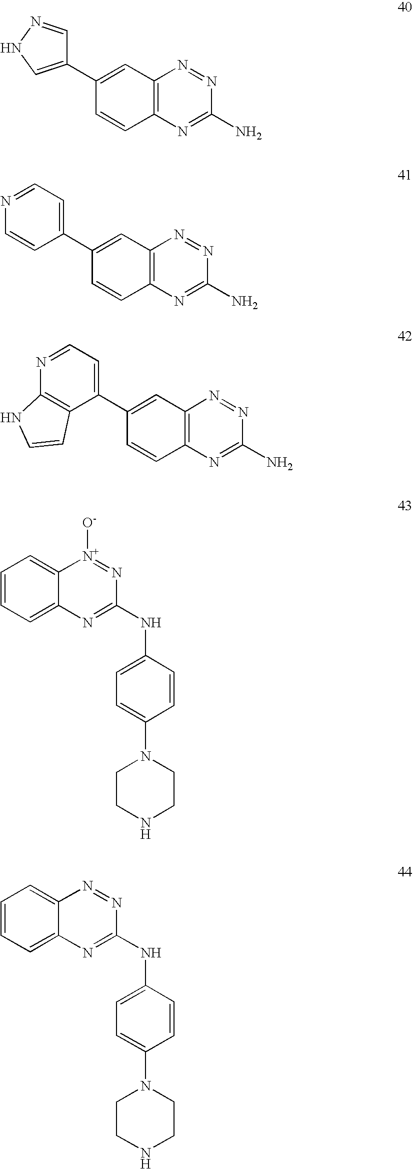

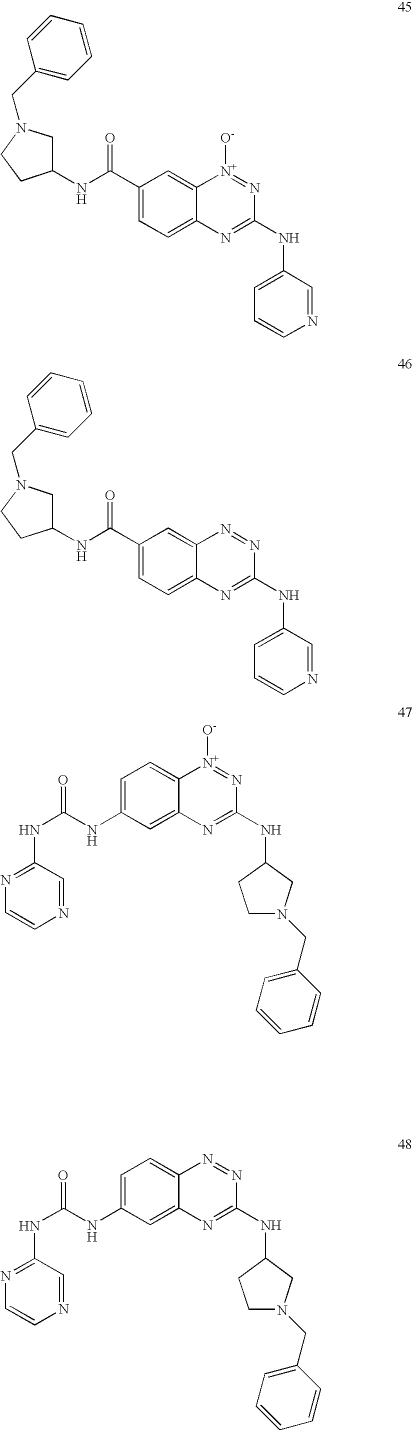

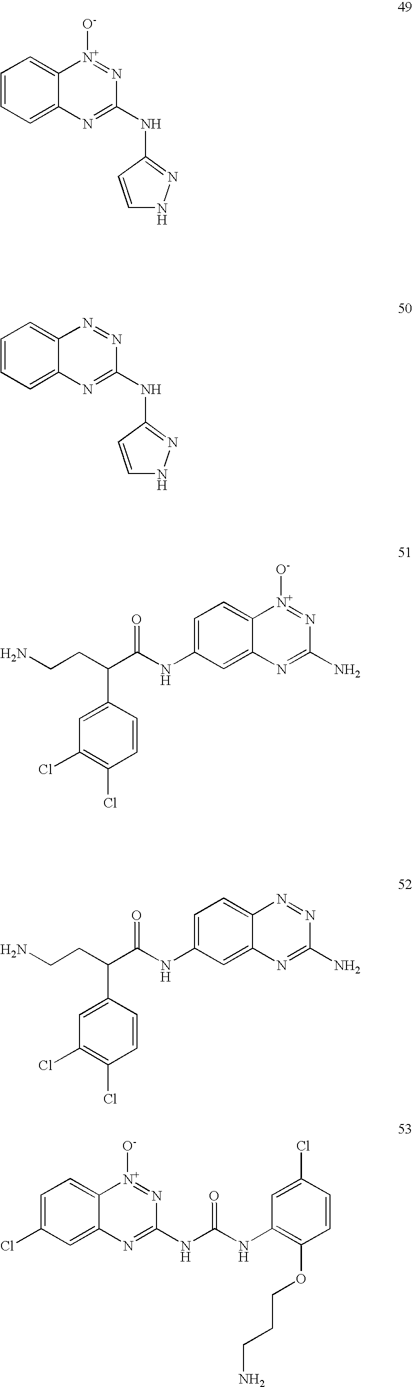

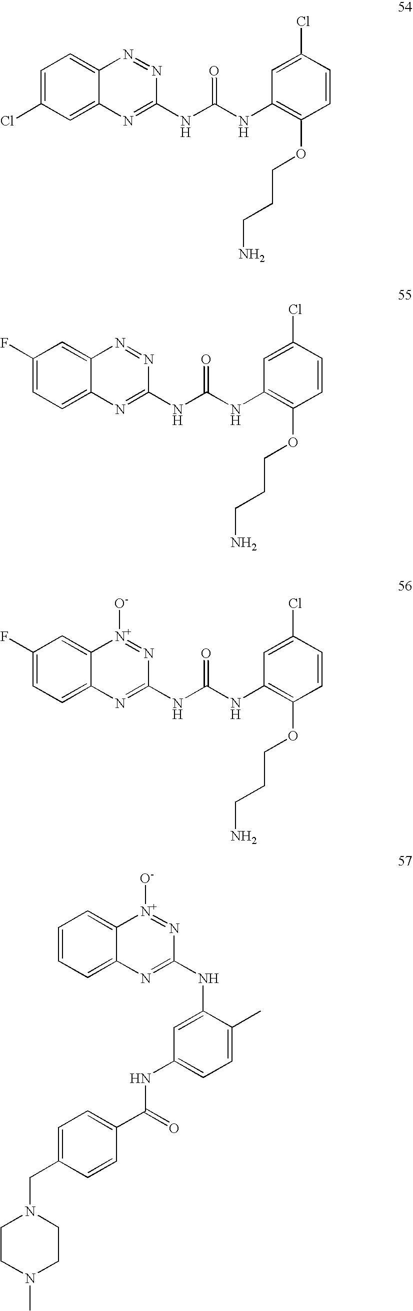

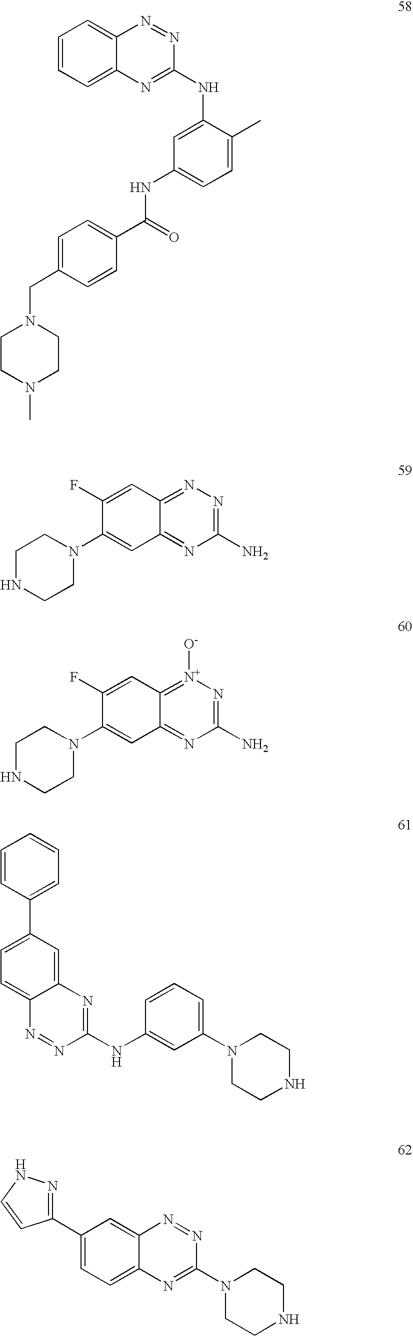

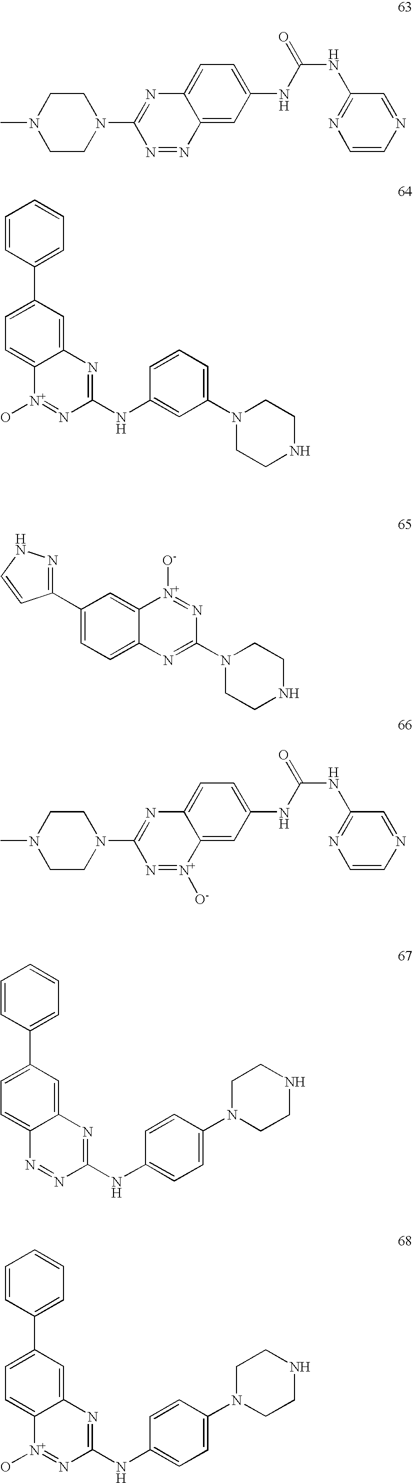

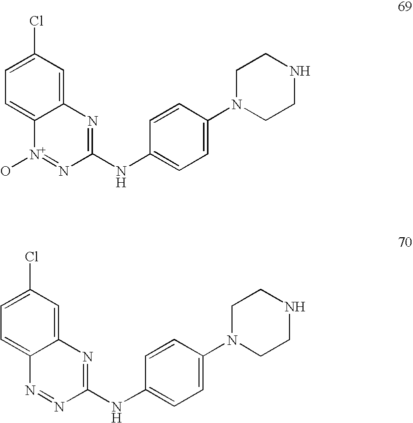

and the dioxides:

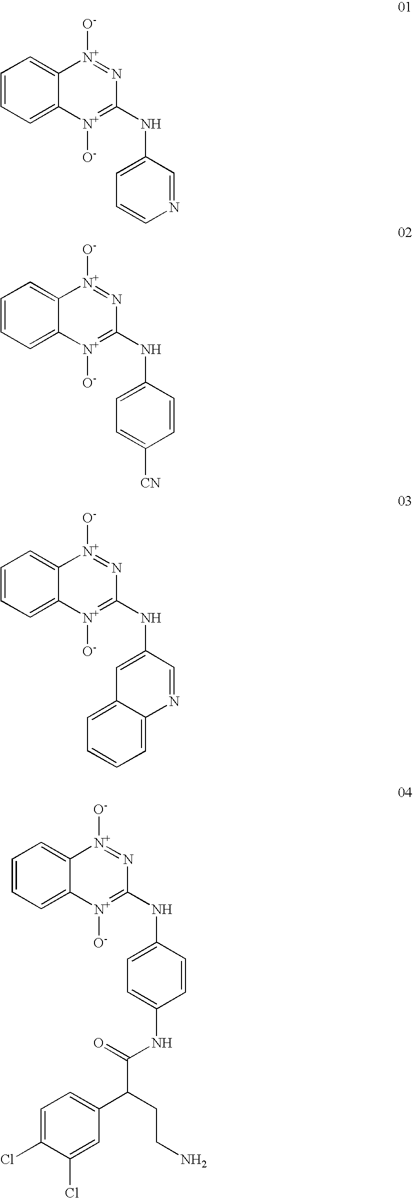

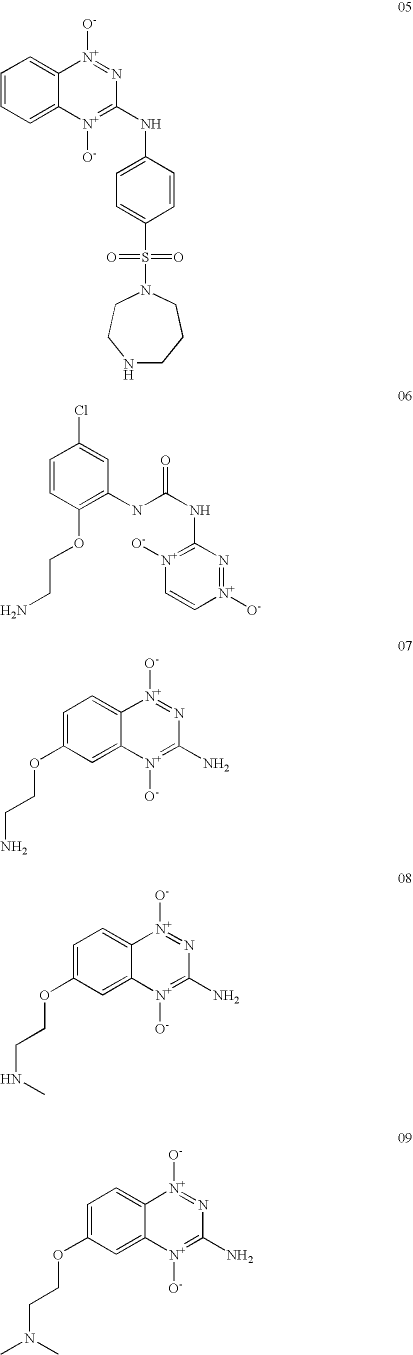

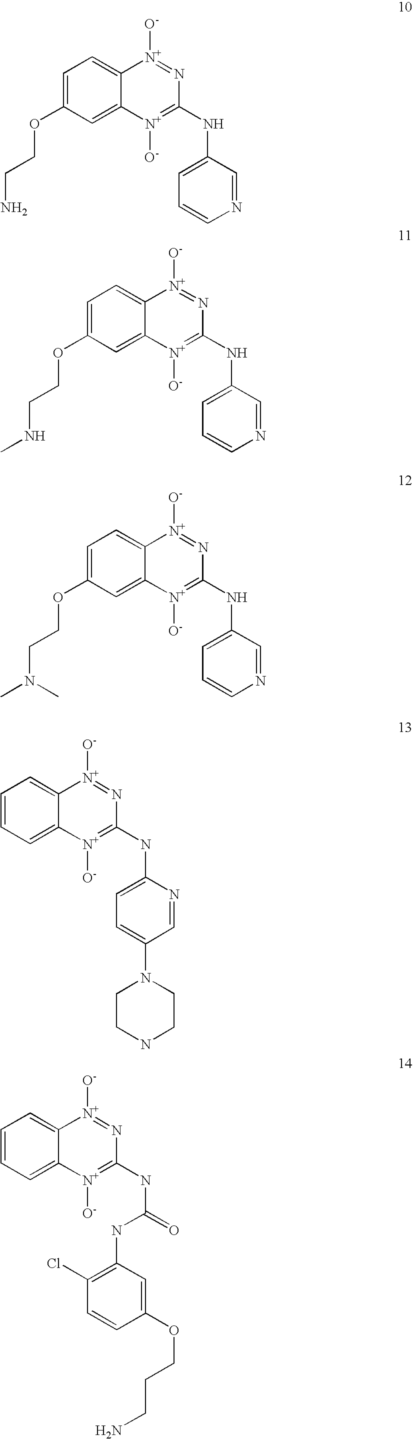

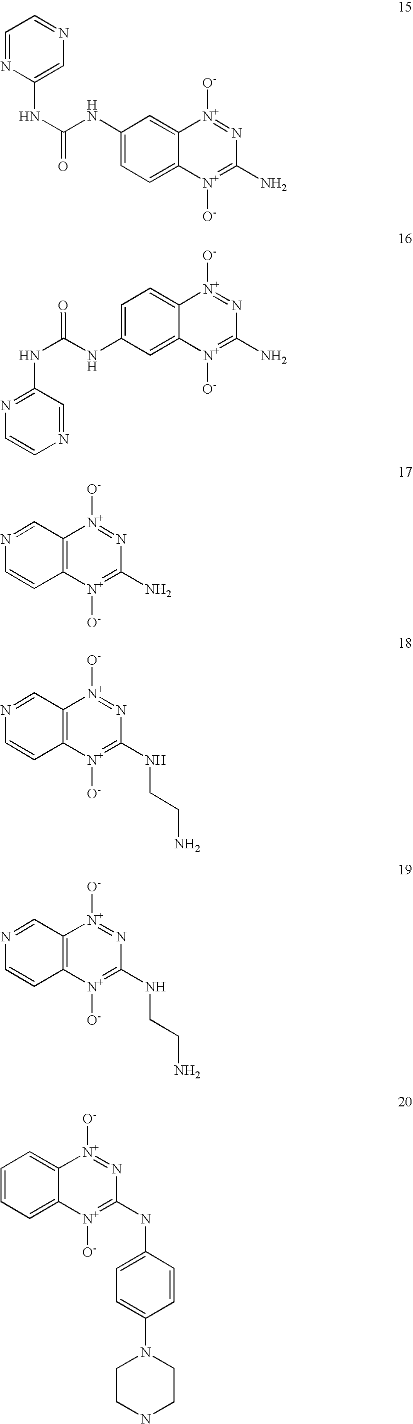

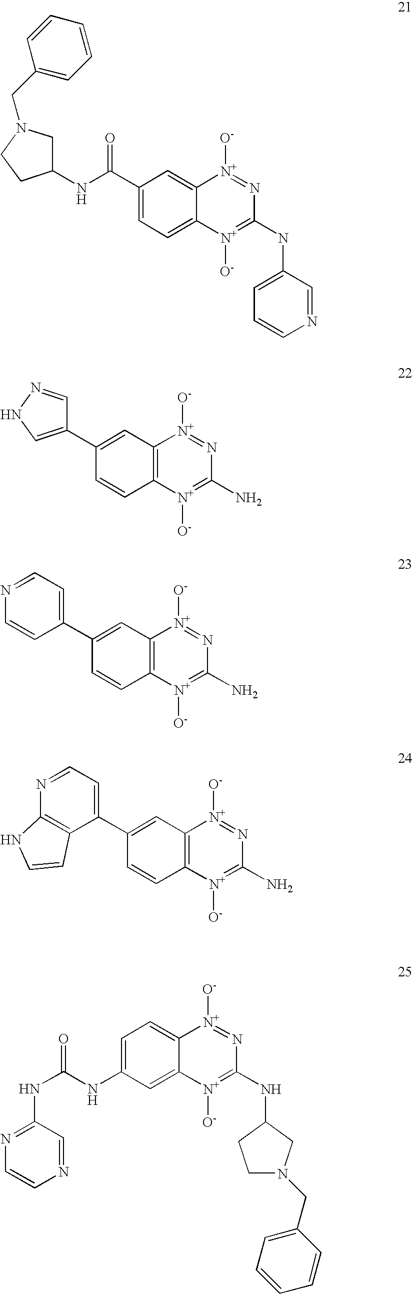

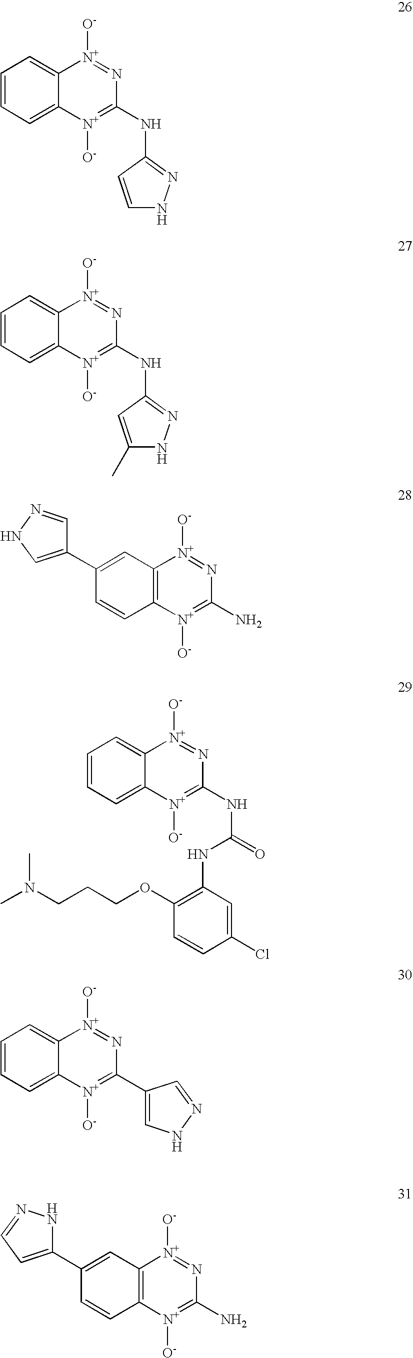

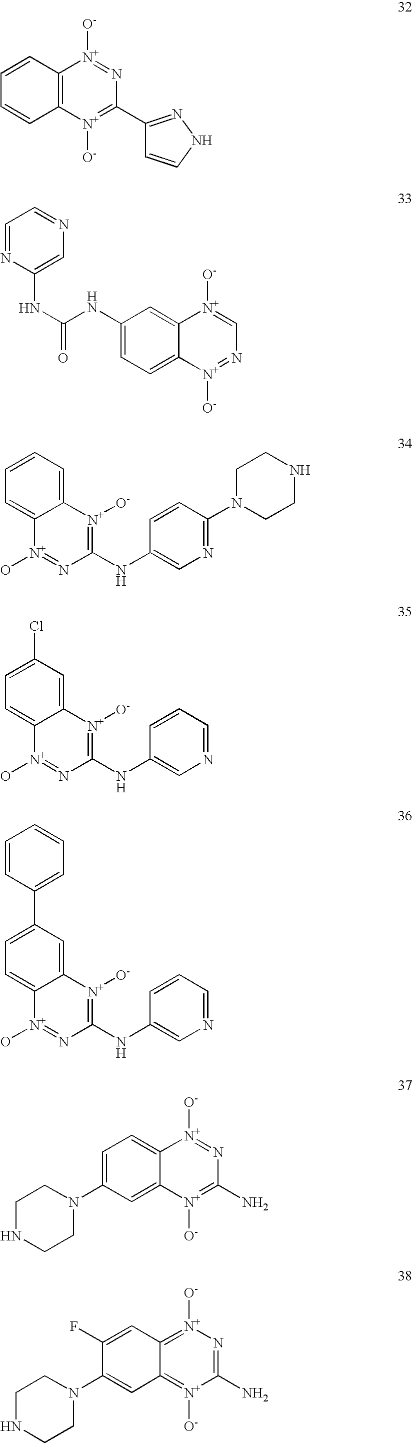

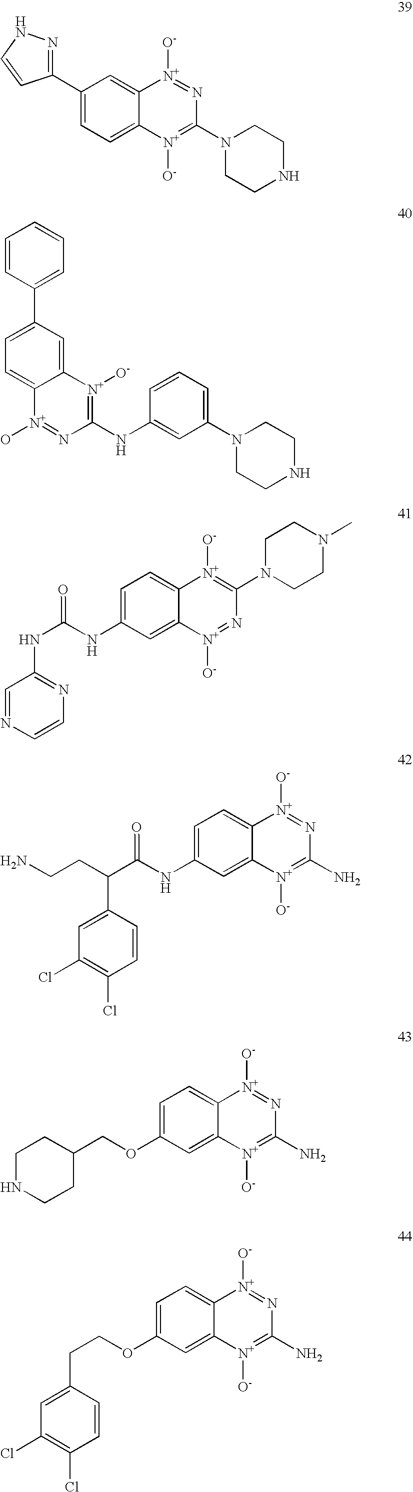

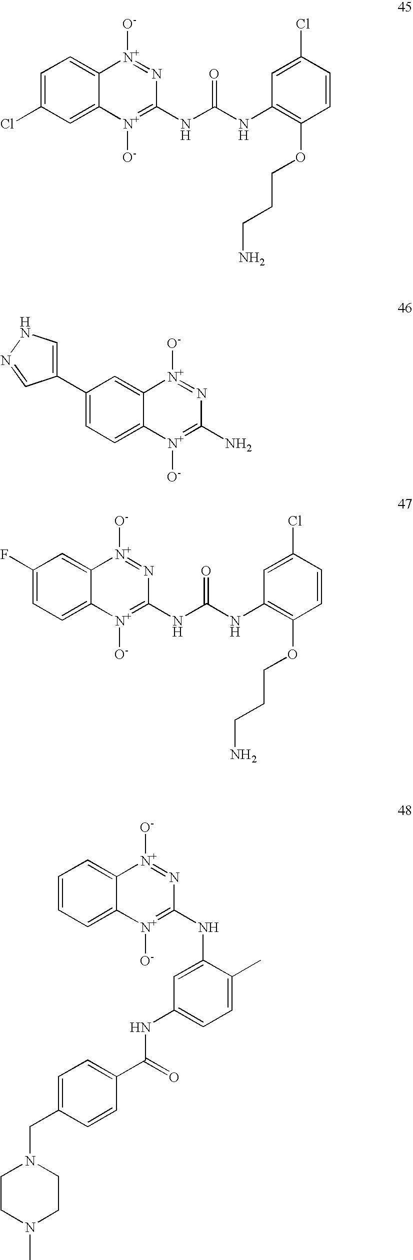

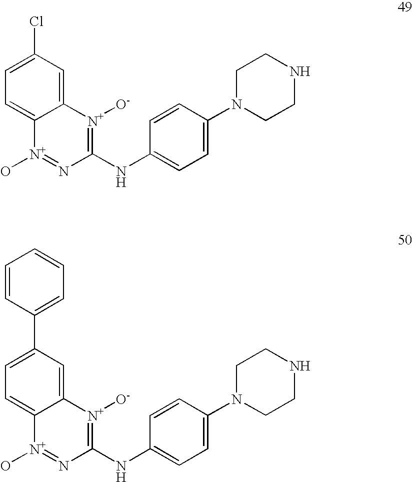

Exemplary methods for preparing compounds of Formula 1 include:

Method (1) Displacement of 3-Chloro-benzo[1,2,4]triazine 1-oxide with various nuclephiles using the conditions previously reported (US2004/0192686A1) will provide the desired products.

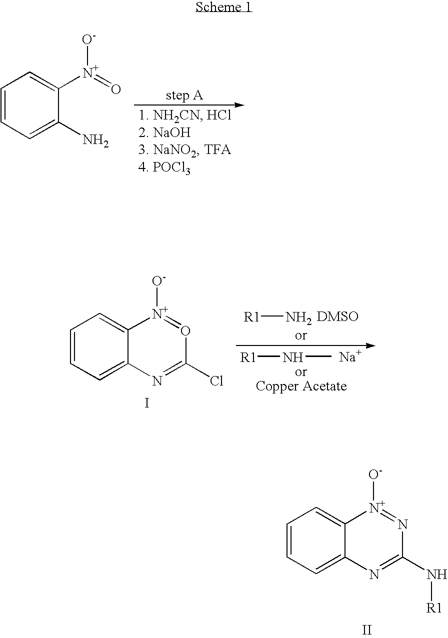

The procedures relating to step A are clearly described in US2004/0192686A1, and are incorporated herein by reference. Exemplary reactants and resulting products are set forth in Table 1.

TABLE 1
Examples of R¹-NH₂ and resulting products

| Amine R1-NH₂ | Product |
|---|---|
| 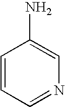 | (1-Oxy-benzo[1,2,4]triazin-3-yl)-pyridin-3-yl-amine |
| 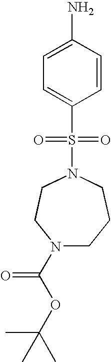 | [4-([1,4]Diazepane-1-sulfonyl)-phenyl]-(1-oxy-benzo[1,2,4]triazin-3-yl)-amine* |
| 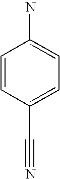 | 4-(1-Oxy-benzo[1,2,4]triazin-3-ylamino)-benzonitrile |
| 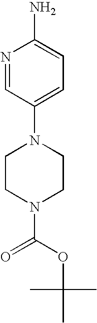 | (1-Oxy-benzo[1,2,4]triazin-3-yl)-(5-piperazin-1-yl-pyridin-2-yl)-amine* |
| 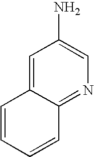 | (1-Oxy-benzo[1,2,4]triazin-3-yl)-quinolin-3-yl-amine |
| 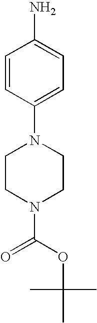 | (1-Oxy-benzo[1,2,4]triazin-3-yl)-(4-piperazin-1-yl-phenyl)-amine* |
| 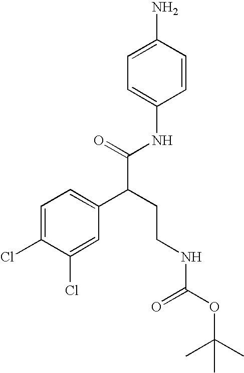 | 4-Amino-2-(3,4-dichloro-phenyl)-N-[4-(1-oxy-benzo[1,2,4]triazin-3-ylamino)-phenyl]-butyramide* |
| 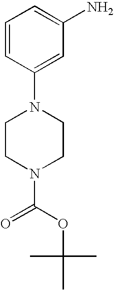 | (1-Oxy-benzo[1,2,4]triazin-3-yl)-(3-piperazin-1-yl-phenyl)-amine |

*The Boc group can be removed under standard acicdic conditions (Hydrochloric acids or trifluoroacetic acid)

Method (2) Following the procedure described in the literature (WO04026846A1), starting from 3-Nitro-pyridin-4-ylamine, 1-Oxy-pyrido[4,3-e][1,2,4]triazin-3 (V) and 3-Chloro-pyrido[4,3-e][1,2,4]triazine 1-oxide-ylamine (VI) can be prepared (scheme 2).

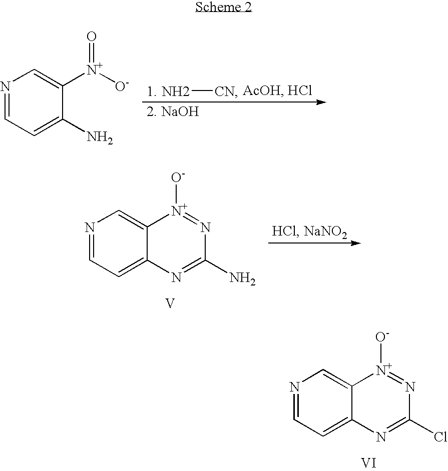

Further, following the conditions outlined in Scheme 1, the chloro group of VI can be replaced with other organic groups.

| Amine R1-NH₂ | Product |
|---|---|
| 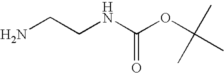 | N*1*-(1-Oxy-pyrido[4,3-e][1,2,4]triazin-3-yl)-ethane-1,2-diamine |

Method (3) Starting with Fluoro-1-oxy-benzo[1,2,4]triazin-3-ylamine (III—scheme 3), SnAr displacent of the fluoride using the same procedure previously reported (US2004/0192686A1) with a range of alcohols (Table 2) will provide the desired products.

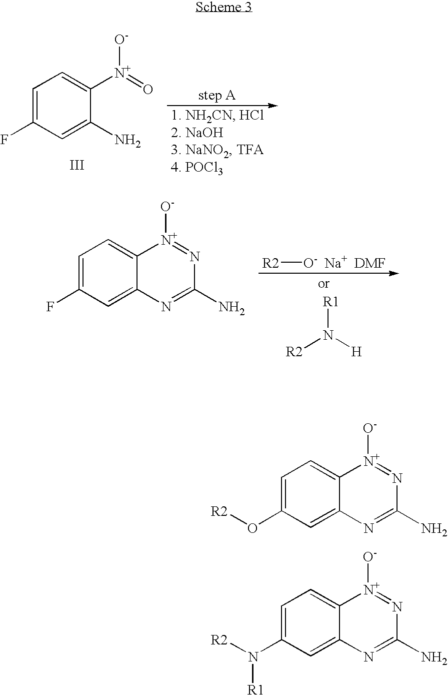

The procedures relating to step A are clearly described in J. Med. Chem. 2003, 46, 169-182, and are incorporated herein by reference.

TABLE 2

| Alcohol^{#} | Product |
|---|---|
| 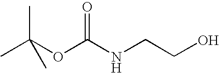 | 6-(2-Amino-ethoxy)-1-oxy-benzo[1,2,4]triazin-3-ylamine* |
| 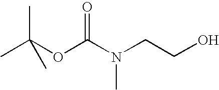 | 6-(2-Methylamino-ethoxy)-1-oxy-benzo[1,2,4]triazin-3-ylamine* |
| 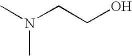 | 6-(2-Dimethylamino-ethoxy)-1-oxy-benzo[1,2,4]triazin-3-ylamine |

*The Boc group can be removed under standard acicdic conditions (Hydrochloric acids or trifluoroacetic acid)
^{#}The sodium salt is prepared from the alcohol with NaH in DMF

Method (4) Following the procedure outlined in US2004/0192686A1, starting with the nitro aniline IV, it is expected that derivatives of 1-Oxy-benzo[1,2,4]triazin-3-ylamine can be prepared (Scheme 4). Further modification by susuki cross coupling from the bromide (A. Suzuki, Synthetic Communications, 11(7), 513-519) or from the chloride (Gregory C. Fu, J. Am. Chem. Soc., 2000, 122, 4020-4028) is expected to yield the final products.

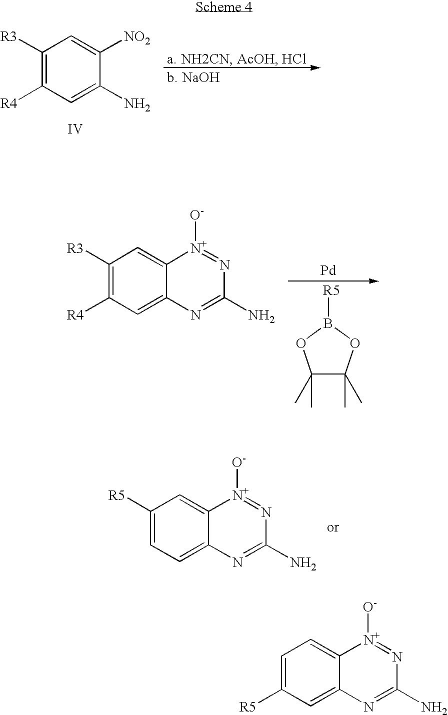

Exemplary reactants and resultant products are set forth in Table 3.

TABLE 3

| Nitro Aniline | Boronic Esters | Product |
|---|---|---|
| 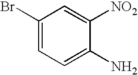 | 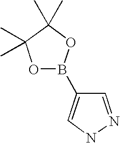 | 1-Oxy-7-(1H-pyrazol-4-yl)-benzo[1,2,4]tri-azin-3-ylamine |
| 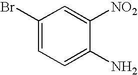 | 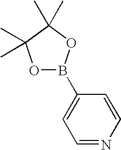 | 1-Oxy-7-pyridin-4-yl-benzo[1,2,4]tri-azin-3-ylamine |
| 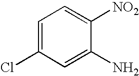 | 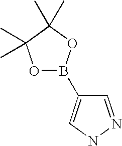 | 1-Oxy-6-(1H-pyrazol-4-yl)-benzo[1,2,4]tri-azin-3-ylamine |
| 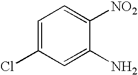 | 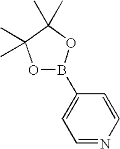 | 1-Oxy-6-pyridin-4-yl-benzo[1,2,4]tri-azin-3-ylamine |

Method (5) In order to generate the parent heterocycle from the N-oxides, reduction of the N-oxides can be achieved by treatment with sodium dithionite in aqueous ethanol following the literature procedure (US2004/0192686A1) as shown in scheme 5. In addition, it is expected that if the parent heterocycle or the mono N-oxide is oxidised with MCPBA, peracetic acid or trifluoroperacetic acid (J. Med. Chem 2003, 46, 169-182), then the corresponding benzotriazine, 1-4 dioxide may be prepared.

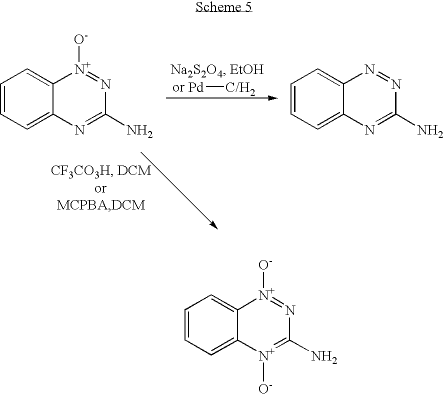

Method (6) In a similar way to the protocols described in scheme 4, the Suzuki cross coupling can be used to prepare 3-substituted benzotriazines and their corresponding dioxides.

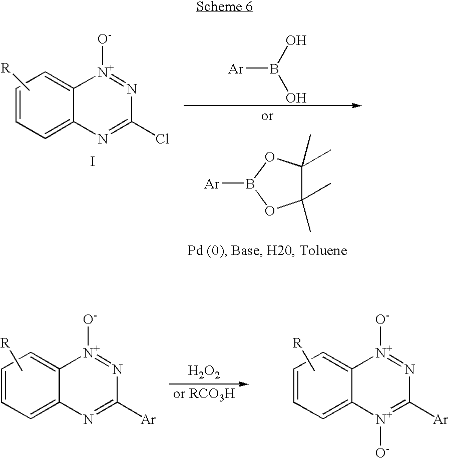

Method (7) In particular, compounds of structure VII can be prepared by the following procedure:

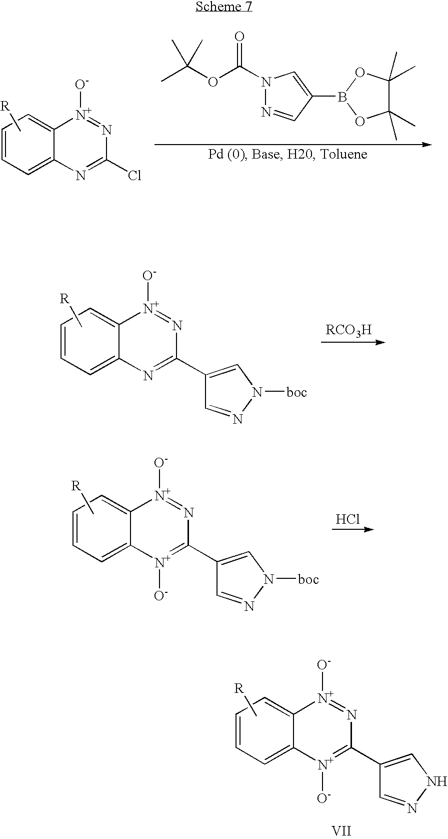

Method (8) Following the same procedure, the following pyrazoles VIII may also be prepared:

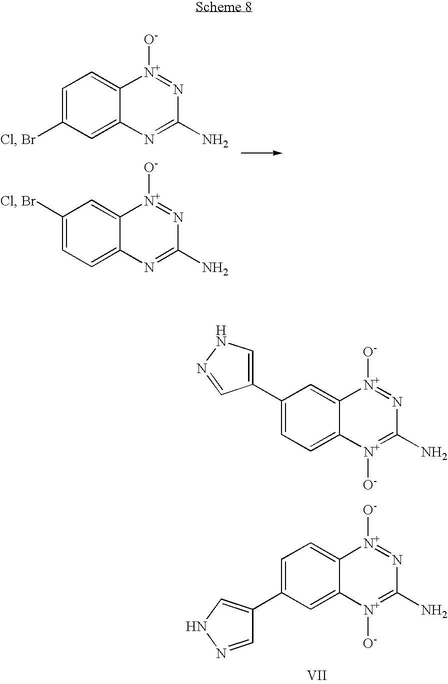

Method (9) Amide derivatives of the benzotriazine can be prepared by the following scheme:

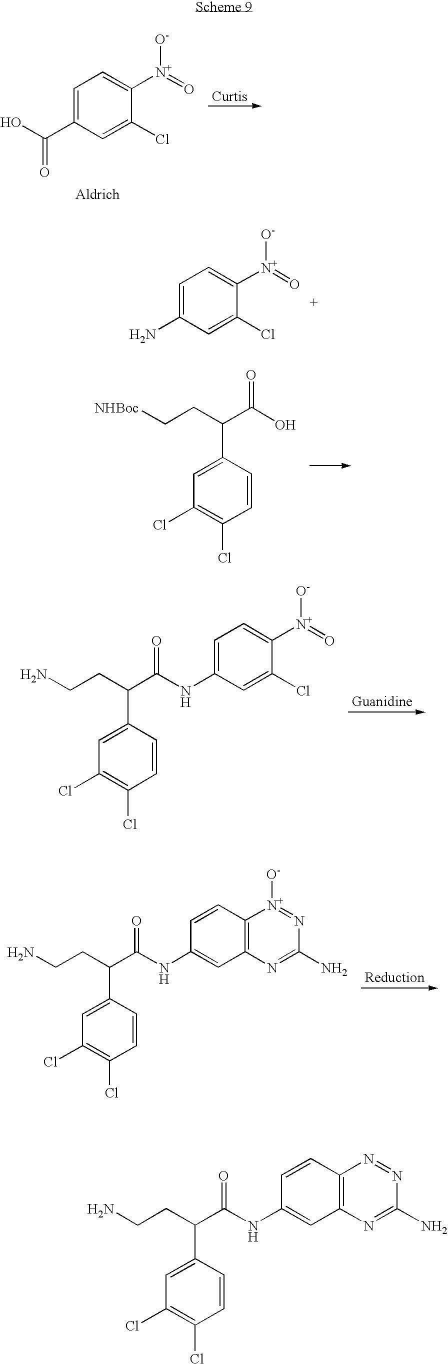

Method (10) Urea modifications can be performed following the protocols outlined below:

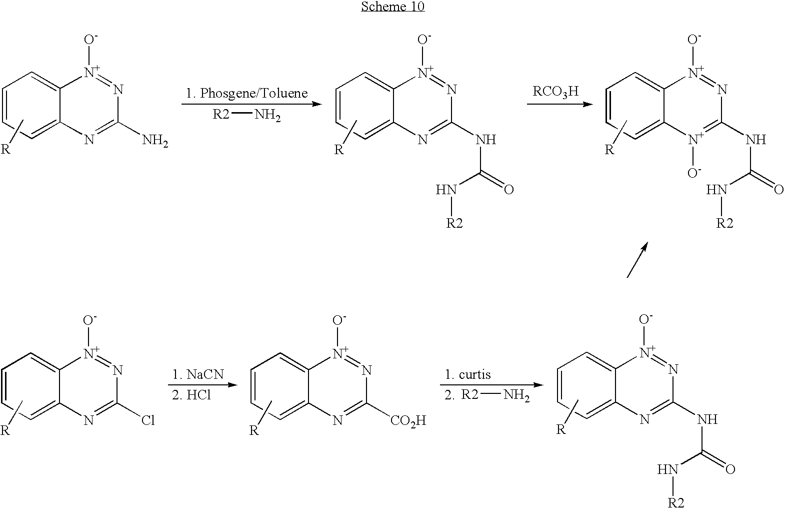

Within the invention it is understood that compounds of Formula I or I(a) may exhibit the phenomenon of tautomerism and that the formula drawings within this specification represent only one of the possible tautomeric forms. It is to be understood that the invention encompasses any tautomeric form which modulates and/or inhibits kinase activity and is not to be limited merely to any one tautomeric form utilized within the formula drawings.

Some of the inventive compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, Formulae I and I(a) are intended to cover solvated as well as unsolvated forms of the identified structures. For example, Formulas I and I(a) include compounds of the indicated structure in both hydrated and non-hydrated forms. Other examples of solvates include the structures in combination with isopropanol, ethanol, methanol, DMSO, ethyl acetate, acetic acid, or ethanolamine.

### Therapeutic Options for the Invention

The term “prodrug” refers to a metabolic precursor of a compound of the Formula I or I(a) (or a salt thereof) that is pharmaceutically acceptable. A prodrug may be inactive when administered to a subject but is converted in vivo to an active compound of the Formula I or I(a). The term “active metabolite” refers to a metabolic product of a compound of the Formula I or I(a) that is pharmaceutically acceptable and effective. Prodrugs and active metabolites of compounds of the Formula I or I(a) may be determined using techniques known in the art. See, e.g., Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); and Larsen, Design and Application of Prodrugs, Drug Design and Development (Krogsgaard-Larsen et al., eds., Harwood Academic Publishers, 1991).

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

The disclosed compounds of Formulae I and I(a) and their pharmaceutically acceptable salts and prodrugs (referred to herein as “the present compounds”) are advantageously administered to inhibit protein kinases in a subject in whom a beneficial therapeutic or prophylactic effect can be achieved by inhibiting protein kinases, i.e., a subject in need of protein kinase inhibition. A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The present compounds can be used to achieve a beneficial therapeutic or prophylactic effect, for example, in subjects with cancer. Cancers which can be treated with the present compounds include solid tumours such as colon, breast, lung, ovarian, pancreatic or non-solid tumours such as non-Hodgkins lymphomas and leukemias

The present compounds are also effective when used in combination with DNA-damaging anti-cancer drugs and/or radiation therapy to treat subjects including those with multi-drug resistant cancers. A cancer is resistant to a drug when it resumes a normal rate of tumour growth while undergoing treatment with the drug after the tumour had initially responded to the drug. A tumour “responds to a drug” when it exhibits a decrease in tumour mass or a decrease in the rate of tumour growth. The term “multi-drug resistant cancer” refers to cancer that is resistant to two or more drugs, typically five or more.

A pharmaceutical composition or preparation according to the invention comprises an effective amount of a protein-kinase modulating agent or a hypoxic selective reducible prodrug therefore, optionally one or more other active agents, and a pharmaceutically acceptable carrier, such as a diluent or excipient for the agent. When the carrier serves as a diluent, it may be a solid, semi-solid, or liquid material acting as a vehicle, excipient, or medium for the active ingredient(s). Compositions according to the invention may be made by admixing the active ingredient(s) with a carrier, or diluting it with a carrier, or enclosing or encapsulating it within a carrier, which may be in the form of a capsule, sachet, paper container, or the like. Exemplary ingredients, in addition to one or more protein kinase modulating agents or prodrug thereore, and any other active ingredients, include Avicel (microcrystalline cellulose), starch, lactose, calcium sulfate dihydrate, terra alba, sucrose, talc, gelatin, agar, pectin, acacia, magnesium stearate, tearic acid, peanut oil, olive oil, glyceryl monostearate, Tween 80 (polysorbate 80), 1,3-butanediol, cocoa butter, beeswax, polyethylene glycol, propylene glycol, sorbitan monostearate, polysorbate 60, 2-octyldodecanol, benzyl alcohol, glycine, sorbic acid, potassium sorbate, disodium hydrogen phosphate, sodium chloride, and water.

The compositions may be prepared in any of a variety of forms suitable for the desired mode of administration. For example, pharmaceutical compositions may be prepared in the form of tablets, pills, powders, lozenges, sachets, cachets, elixirs, suspensions, emulsions, solutions, syrups, aerosols (as solids or in liquid media), ointments (e.g., containing up to 10% by weight of a protein kinase modulating agent), soft-gel and hard-gel capsules, suppositories, sterile injectable solutions, sterile packaged powders, and the like.

Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can thus be employed. If a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent may be dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, gylcerin and the like in concentrations ranging from 0-60% of the total volume. A compound of Formula I or I(a) may be dissolved in DMSO and diluted with water. The composition may also be in the form of a solution of a salt form of the active ingredient in an appropriate aqueous vehicle such as water or isotonic saline or dextrose solution.

Therapeutically effective amounts of the agents of the invention may be used to treat diseases mediated by modulation or regulation of protein kinases. An “effective amount” is intended to mean that amount of an agent that, when administered to a mammal in need of such treatment, is sufficient to effect treatment for a disease mediated by the activity of one or more kinases. Thus, e.g., a therapeutically effective amount of a compound of the Formula I or II, salt, active metabolite or prodrug thereof is a quantity sufficient to modulate, regulate, or inhibit the activity of one or more kinases such that a disease condition which is mediated by that activity is reduced or alleviated.

“Treating” is intended to mean at least the mitigation of a disease condition in a mammal, such as a human, that is affected, at least in part, by the activity of one or more kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

The amount of the present compounds administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used anti-cancer drugs and radiation therapy are well known to the skilled person. Effective amounts of the present compounds typically range between about 1 mg/m² per day and about 10 grams/m² per day, and preferably between 10 mg/m² per day and about 5 grams/m².

Techniques for formulation and administration of the compounds of the instant invention can be found in Remington: the Science and Practice of Pharmacy, 19^{th} edition, Mack Publishing Co., Easton, Pa. (1995). The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/ciliary, lens, choroid/retina and sclera. The pharmaceutically acceptable ophthalmic vehicle may be an ointment, vegetable oil, or an encapsulating material. A compound of the invention may also be injected directly into the vitreous and aqueous humor.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

The compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

Preferably disclosed compounds or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient (viz., a compound of Structural Formula I or II or salts or prodrugs thereof) in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

### EXAMPLES

### Preparation of Heterocyclic Triazine Compounds of the Invention.

### Examples 1 and 2

1-Oxy-benzo[1,2,4]triazin-3-ylamine and Benzo[1,2,4]triazin-3-ylamine were prepared following the method published in J. Med. Chem. 2003, 46, 169-182

### Example 3

### Benzo[1,2,4]triazin-3-yl-pyridin-3-yl-amine

To a solution of (1-Oxy-benzo[1,2,4]triazin-3-yl)-pyridin-3-yl-amine (example 4, 0.5 g) in ethanol (10 ml) and water (3 ml) was added sodium dithionate (1.09 g) portion wise over a period of 1 h. The reaction mixture was refluxed for 2 h. On completion, the compound was extracted with ethylacetate (50 ml), washed with water (30 ml) and dried over sodium sulfate (2 g). Crude material purified by column chromatography (SiO₂, 40% EtOAc/n-hexane) to yield the desired product (0.35g, 76%).

### Example 4

### (1-Oxy-benzo[1,2,4]triazin-3-yl)-pyridin-3-yl-amine

3-Chloro-benzo[1,2,4]triazine 1-oxide was prepared according to the literature procedure (US20040192686A1). 3-aminopyridine (2.3 g) in dry DMF (50 ml) was cooled to 0-5° C. and NaH (1.78 g) added portion wise over a period of 0.5 h. The reaction mixture was stirred at room temperature for 15 min., then this mixture was added to 3-Chloro-benzo[1,2,4]triazine 1-oxide (4.5 g) and stirred at room temperature for 2 h. On completion, the compound was extracted with ethylacetate (500 ml), washed with water (1000 ml) and dried over sodium sulfate (10 g). The solvent was removed under reduced pressure and residue purified by column chromatography (SiO₂, 50% EtOAc/n-hexane) to yield (1-Oxy-benzo[1,2,4]triazin-3-yl)-pyridin-3-yl-amine (2.5 g, 42% yield).

### Example 5

### (1-Oxy-benzo[1,2,4]triazin-3-yl)-(4-piperazin-1-yl-phenyl)-amine

4-(4-Amino-phenyl)-piperazine-1-carboxylic acid tert-butyl ester (1 g), 3-Chloro-benzo[1,2,4]triazine 1-oxide (0.65 g), potassium carbonate (0.64 g), N,N-Dimethyl-formamide (15 ml) and copper acetate (0.1 g) were heated to 85° C. for 6 h. On completion, the reaction mixture was allowed to warm to RT and water (100 ml) was added. The reaction mixture was extracted with ethyl acetate (3×50 ml). The combined organic extracts were washed with water (3×50 ml) and dried over sodium sulfate. The solvent was removed under reduced pressure to give a residue, which was further purified by column chromatography (SiO₂, 25% EtOAc/n-hexane) yielding 4-[4-(1-Oxy-benzo[1,2,4]triazin-3-ylamino)-phenyl]-piperazine-1-carboxylic acid tert-butyl ester in 31% yield. This product (0.62 g) was dissolved in chloroform (9 ml), cooled to 0° C. and dry hydrogen chloride gas was bubbled through the reaction mixture for a period of 30 minutes. On completion, the reaction mixture was basified with 5% NaOH saturated with NaCl (˜40 ml) to pH˜9 at 0° C. The reaction mixture was filtered and the organic layer was separated. The organic layer was dried over sodium sulfate. The solvent was removed under reduced pressure to give a residue, which was washed with hexane (˜20 ml) and ethyl acetate (˜20 ml) to give the desired product (1-Oxy-benzo[1,2,4]triazin-3-yl)-(4-piperazin-1-yl-phenyl)-amine in 51% yield.

### Example 6

### Benzo[1,2,4]triazin-3-yl-(4-piperazin-1-yl-phenyl)-amine

Prepared following the procedure outlined in example 3.

### Example 7

### 7-Bromo-1-oxy-benzo[1,2,4]triazin-3-ylamine

4-Bromo-2-nitroaniline (2 g, 9.2 mmol) and cyanamide (1.97 g, 47 mmol) were melted together at 100° C. The reaction mixture was then cooled to around 50° C. and conc. HCl (17 ml) was added carefully over a period of 30 minutes, maintaining the temperature at 50° C. The reaction mixture was stirred at this temperature until the exotherm subsided and then stirred at 100° C. for 4 hr. The reaction mixture was cooled to room temperature and made strongly basic with 7.5 M NaOH (160 ml) and then stirred at 100° C. for 1 hr. It was cooled to room temperature and diluted with water (100 ml). The precipitate formed was filtered, washed with water, ether and dried to yield 1.2 g of 7-Bromo-1-oxy-benzo[1,2,4]triazin-3-ylamine, as a yellow solid.

### 1-Oxy-7-(1H-pyrazol-4-yl)-benzo[1,2,4]triazin-3-ylamine

To a degassed solution of 7-Bromo-1-oxy-benzo[1,2,4]triazin-3-ylamine (0.3 g, 1.24 mmol), pyrazole-4-boronic acid pinacol ester (0.28 g, 1.5 mmol) and CsCO₃ (1.2 g, 3.7 mmol) in DME (30 ml)/H₂O (10 ml) was added tetrakis(triphenylphosphine)palladium(0) (0.071 g, 0.06 mmol). The reaction mixture was stirred at 100° C. overnight (under argon) and extracted with ethyl acetate. The combined organic layers were dried over anhydrous sodium sulfate, concentrated to get the crude product, which was purified by column chromatography (silica gel 60-120, CHCl₃/MeOH) to yield 0.200 g of 1-Oxy-7-(1H-pyrazol-4-yl)-benzo[1,2,4]triazin-3-ylamine, as a yellow solid.

### Example 8

### 7-(1H-Pyrazol-4-yl)-benzo[1,2,4]triazin-3-ylamine

To a solution of 1-Oxy-7-(1H-pyrazol-4-yl)-benzo[1,2,4]triazin-3-ylamine (0.10 g) in MeOH (10 ml) was added 10% Pd/C (0.02 g) and the reaction mixture was stirred under H₂ (balloon) at room temperature for 1 hr. After completion of reaction (TLC), the catalyst was filtered off and washed with methanol. The combined filtrate was concentrated to get the crude product, which was purified by column chromatography (silica gel, CHCl₃/MeOH) to get 0.03 g of 7-(1H-Pyrazol-4-yl)-benzo[1,2,4]triazin-3-ylamine, as a yellow solid.

### Example 9

### 1-Benzo[1,2,4]triazin-3-yl-3-[5-chloro-2-(3-dimethylamino-propoxy)-phenyl]-urea

5-Chloro-2-(3-dimethylamino-propoxy)-phenylamine was prepared following procedure outlined in WO03101444. Benzo[1,2,4]triazin-3-ylamine was prepared as described in example 2. Benzo[1,2,4]triazin-3-ylamine (100 mg) was suspended in 5 ml dichloromethane and 2.0 equivalents of triethylamine were added followed by the addition of 0.452 mmoles of triphosgene. The mixture was stirred at room temperature for 30 minutes after which 0.68 mmol of 5-Chloro-2-(3-dimethylamino-propoxy)-aniline was added. The reaction mixture was stirred for a further 30 minutes. The product is purified by column chromatography (triethylamine/methanol/dichloromethane=1/9/90).

### Example 10

### (5-Methyl-1H-pyrazol-3-yl)-(1-oxy-benzo[1,2,4]triazin-3-yl)-amine

5-Methyl-3-aminopyrazole (0.53 g) and N-methyl pyrrolidone (5 ml) were stirred under nitrogen. Copper powder (0.08 g), copper iodide (0.2 g) and potassium carbonate (1.1 g) were added. The reaction mixture was stirred at RT for 10 min. Then 3-Chloro-benzo[1,2,4]triazine 1-oxide (0.5 g) was added to the above reaction mixture and heated to 55-60° C. The reaction mixture was stirred for 30 min at 55-60° C. On completion, water (100 ml) was added to the reaction mixture and extracted with ethyl acetate (3×100 ml). The combined organic extracts were dried over sodium sulfate. The solvent was removed under reduced pressure to give a residue, which was further purified by column chromatography (SiO₂, 60% EtOAc/n-hexane) to yield (5-Methyl-1H-pyrazol-3-yl)-(1-oxy-benzo[1,2,4]triazin-3-yl)-amine in 6% yield.

### Example 11

### Benzo[1,2,4]triazin-3-yl-(5-methyl-1H-pyrazol-3-yl)-amine

Prepared following the procedure outlined in example 3 starting from (5-Methyl-1H-pyrazol-3-yl)-(1-oxy-benzo[1,2,4]triazin-3-yl)-amine.

### Example 12

### 3-(1H-Pyrazol-4-yl)-benzo[1,2,4]triazine 1-oxide

To a degassed solution of 3-Chloro-benzo[1,2,4]triazine 1-oxide (0.4 g, 2.2 mmol), 4-(4,4,5,5-Tetramethyl-[1,3,2]dioxaborolan-2-yl)-1H-pyrazole (0.51 g, 2.6 mmol) and CsCO₃ (2.15 g, 6.6 mmol) in DME (30 ml)/H₂O (10 ml) was added tetrakis(triphenylphosphine)palladium(0) (0.12 g, 0.11 mmol). The reaction mixture was stirred at 80° C. for 2 hr (under argon) and extracted with ethyl acetate. The combined organic layers were dried over anhydrous sodium sulfate, concentrated to get the crude product, which was purified by column chromatography (silica gel 60-120, CHCl₃/MeOH) and preparative HPLC to get 0.03 g of 3-(1H-Pyrazol-4-yl)-benzo[1,2,4]triazine 1-oxide as a yellow solid.

### Example 13

### 3-(1H-Pyrazol-4-yl)-benzo[1,2,4]triazine

To a solution of 3-(1H-Pyrazol-4-yl)-benzo[1,2,4]triazine 1-oxide (0.10 g) in MeOH (10 ml) was added 10% Pd/C (0.02 g) and the reaction mixture was stirred under H₂ (balloon) at room temperature for 1 hr. After completion of reaction, the catalyst was filtered off and washed with methanol. The combined filtrate was concentrated to get the crude product, which was purified by column chromatography (silica gel, CHCl₃/MeOH) to get 0.03 g of 3-(1H-Pyrazol-4-yl)-benzo[1,2,4]triazine as a yellow solid.

### Example 14

### 7-(2H-Pyrazol-3-yl)-benzo[1,2,4]triazin-3-ylamine

To a solution of 1-Oxy-7-(2H-pyrazol-3-yl)-benzo[1,2,4]triazin-3-ylamine (0.10 g) in MeOH (10 ml) was added 10% Pd/C (0.02 g) and the reaction mixture was stirred under H₂ (balloon) at room temperature for 1 hr. After completion of reaction, the catalyst was filtered off and washed with methanol. The combined filtrate was concentrated to get the crude product, which was purified by column chromatography (silica gel, CHCl₃/MeOH) to get 0.04 g of 7-(2H-Pyrazol-3-yl)-benzo[1,2,4]triazin-3-ylamine, as a yellow solid.

### Example 15

### 1-Oxy-7-(2H-pyrazol-3-yl)-benzo[1,2,4]triazin-3-ylamine

To a degassed solution of 7-Bromo-1-oxy-benzo[1,2,4]triazin-3-ylamine (0.4 g, 1.66 mmol), 1-(Tetrahydro-pyran-2-yl)-1H-Pyrazol-3-yl-boronic acid (0.485 g, 2.5 mmol) and CsCO₃ (1.82 g, 5.6 mmol) in DME (30 ml)/H₂O (10 ml) was added tetrakis(triphenylphosphine)palladium(0) (0.095 g, 0.08 mmol). The reaction mixture was stirred at 100° C. overnight (under argon) and extracted with ethyl acetate. The combined organic layers were dried over anhydrous sodium sulfate and concentrated to give the crude product, which was purified by column chromatography (silica gel, CHCl₃/MeOH) to yield 1-Oxy-7-[2-(tetrahydro-pyran-2-yl)-2H-pyrazol-3-yl]-benzo[1,2,4]triazin-3-ylamine. This was stirred with ethereal Hydrogen Chloride (10 ml) at room temperature for 30 min. The reaction mixture was neutralised with Na₂CO₃ and extracted with ethyl acetate. The solvent was evaporated and the residue was washed with ether to get 0.125 g of 1-Oxy-7-(2H-pyrazol-3-yl)-benzo[1,2,4]triazin-3-ylamine, as a yellow solid.

### Example 16

### 3-(1H-Pyrazol-3-yl)-benzo[1,2,4]triazine

To a solution of 3-(1H-Pyrazol-3-yl)-benzo[1,2,4]triazine 1-oxide (0.10 g) in MeOH (10 ml) was added 10% Pd/C (0.02 g) and the reaction mixture was further stirred under H₂ (balloon) at room temperature for 1 hr. After completion of reaction, the catalyst was filtered off and washed with methanol. The combined filtrate was concentrated to get the crude product, which was purified by column chromatography (silica gel, CHCl₃/MeOH) to give 0.03 g of 3-(1H-Pyrazol-3-yl)-benzo[1,2,4]triazine, as a yellow solid.

### Example 17

### 3-(1H-Pyrazol-3-yl)-benzo[1,2,4]triazine 1-oxide

To a degassed solution of 3-Chloro-benzo[1,2,4]triazine 1-oxide (0.2 g, 1.1 mmol), 1-(Tetrahydro-pyran-2-yl)-1H-Pyrazol-3-yl-boronic acid (0.32 g, 1.6 mmol) and CsCO₃ (1.07 g, 3.3 mmol) in DME (10 ml)/H₂O (5 ml) was added tetrakis(triphenylphosphine)palladium(0) (0.063 g, 0.05 mmol). The reaction mixture was stirred at 100° C. overnight (under argon) and extracted with ethyl acetate. The combined organic layers were dried over anhydrous sodium sulfate, concentrated to get the crude product, which was purified by column chromatography (silica gel, CHCl₃/MeOH) to give 0.1 g of 3-[2-(Tetrahydro-pyran-2-yl)-2H-pyrazol-3-yl]-benzo[1,2,4]triazine 1-oxide. This was stirred with HCl/ether (10 ml) at room temperature for 30 min. The reaction mixture was neutralised with Na₂CO₃ and extracted with ethyl acetate. The solvent was evaporated and the residue was washed with ether to get 0.07 g of 3-(1H-Pyrazol-3-yl)-benzo[1,2,4]triazine 1-oxide, as a yellow solid.

### Example 18

### 6-Fluoro-1-oxy-benzo[1,2,4]triazin-3-ylamine

The title compound was prepared from 1,4-Difluoro-2-nitro-benzene and guanidine following the protocol outlined in J. Med. Chem. 2003, 46, 169-182 and US2004/0192686.

### N*6*-(2,4-Dimethoxy-benzyl)-1-oxy-benzo[1,2,4]triazine-3,6-diamine

6-Fluoro-1-oxy-benzo[1,2,4]triazin-3-ylamine (1.0 g) was dissolved in 10 ml DMSO and 1.2 equivalents of 2,4-Dimethoxy-benzylamine added. The reaction was heated at 100 degree for 2 days. Upon consumption of the starting material, the reaction mixture was cooled to room temperature and water was added to precipitate the desired product that was filtered and washed with plenty of water to afford yellow solid.

### 1-Oxy-benzo[1,2,4]triazine-3,6-diamine

N*6*-(2,4-Dimethoxy-benzyl)-1-oxy-benzo[1,2,4]triazine-3,6-diamine (0.35 g) was dissolved in 5 ml trifluoroacetic acid (TFA) and the reaction mixture was stirred at room temperature for 3 hours. Upon completion, the TFA salt was precipitated from ether and filtered, washed with ether and dried giving 420 mg of desired compound as the TFA salt.

### 1-(3-Amino-1-oxy-benzo[1,2,4]triazin-6-yl)-3-pyrazin-2-yl-urea

Pyrazine-2-carbonyl azide (0.25 g) was dissolved in 2 ml dichloromethane and microwaved at 120° C. for 5 minutes. 1-Oxy-benzo[1,2,4]triazine-3,6-diamine was added to the solution in DMSO (61 mg in 2 ml) and the resulting reaction mixture was heated at 120° C. for a further 10 minutes. The desired product was precipitated with water and the dichloromethane was removed by evaporation. The precipitate was filtered and washed with water and ethanol and subsequently purified by column using 10% saturated NH₃/MeOH in dichloromethane.

### 1-(3-Amino-benzo[1,2,4]triazin-6-yl)-3-pyrazin-2-yl-urea

1-(3-Amino-1-oxy-benzo[1,2,4]triazin-6-yl)-3-pyrazin-2-yl-urea (100 mg) was suspended in 50 mls of 70% ethanol and 2.0 equivalents of Na₂S₂O₄ were added. The resulting reaction mixture was refluxed overnight and the product was purified by column chromatography eluting 10% saturated NH₃/MeOH and 90% dichloromethane (DCM).

### 1-Benzo[1,2,4]triazin-6-yl-3-pyrazin-2-yl-urea

1-(3-Amino-benzo[1,2,4]triazin-6-yl)-3-pyrazin-2-yl-urea (70 mg) was dissolved in 10 ml of DMF and 5.0 equivalents of isoamylnitrite were added and the resulting reaction mixture was heated to 60° C. for 1 hour. The desired product was purified by column chromatography eluting 0-15% sat. NH₃/methanol in DCM.

### Example 19

### Benzo[1,2,4]triazin-3-yl-(6-piperazin-1-yl-pyridin-3-yl)-amine

(1-Oxy-benzo[1,2,4]triazin-3-yl)-(6-piperazin-1-yl-pyridin-3-yl)-amine (20 mg), acetic acid (6 ml), sodium dithionite (25 mg) and water (2 ml) were heated to 100° C. for 30 min. Further sodium dithionite (10 mg) was added to the reaction mixture at 100° C. and stirring continued for a further 30 minutes whereby the remaining sodium dithionite (5 mg) was added and the reaction mixture was stirred at 100° C. for a final 2 hrs. On completion, the solvent was removed under reduced pressure and water (100 ml) was added. The reaction mixture was basified with sodium carbonate solution to pH˜8. The reaction mixture was extracted with chloroform: IPA (80 ml: 20 ml) and the organic layer was dried over sodium sulfate. The solvent was removed under reduced pressure to give a residue, which was washed with ethyl acetate to give the title compound.

### Example 20

### 4-(5-Nitro-pyridin-2-yl)-piperazine-1-carboxylic acid tert-butyl ester

Potassium carbonate (5 g) and Cu(OAc)₂.2H₂O (0.4 g) were added to a solution of piperazine-1-carboxylic acid tert-butyl ester (5 g) and 2-Chloro-5-nitropyridine (4.5 g) in DMF (75 ml) and the reaction mixture was stirred at 85° C. for 3 h. On completion, the reaction mixture was allowed to cool to RT and the DMF was removed under reduced pressure. Water (200 ml) was added to the reaction mixture resulting in the formation of a solid that was filtered and dissolved in ethyl acetate (400 ml). The organic layer was washed with water (200 ml) and dried over sodium sulfate. The solvent was removed under reduced pressure to give 4-(5-Nitro-pyridin-2-yl)-piperazine-1-carboxylic acid tert-butyl ester.

### 4-(5-Amino-pyridin-2-yl)-piperazine-1-carboxylic acid tert-butyl ester

4-(5-Nitro-pyridin-2-yl)-piperazine-1-carboxylic acid tert-butyl ester (5 g), 10% Pd/C (0.5 g) and ethanol (200 ml) were taken together in a hydrogenation flask and the reaction mixture was hydrogenated at 60 psi for 2 hrs. On completion, the reaction mixture was filtered through a celite bed and the ethanol was removed under reduced pressure to give the crude product. The compound was further purified by crystallization from 50% EtOAc/n-hexane (200 ml) to yield 4-(5-Amino-pyridin-2-yl)-piperazine-1-carboxylic acid tert-butyl ester.

### 4-[5-(1-Oxy-benzo[1,2,4]triazin-3-ylamino)-pyridin-2-yl]-piperazine-1-carboxylic acid tert-butyl ester

4-(5-Amino-pyridin-2-yl)-piperazine-1-carboxylic acid tert-butyl ester (1.5 g), 3-Chloro-benzo[1,2,4]triazine 1-oxide (1.2 g) and DMF (20 ml) and potassium carbonate (1.5 g) were heated to 50-60° C. and stirred over night. On completion, water (200 ml) was added and extracted with ethyl acetate (2×200 ml). The combined organic extracts were dried over sodium sulfate. The solvent was removed under reduced pressure to give a residue, which was further purified by column chromatography (SiO₂, 25% EtOAc/n-hexane) to yield 4-[5-(1-Oxy-benzo[1,2,4]triazin-3-ylamino)-pyridin-2-yl]-piperazine-1-carboxylic acid tert-butyl ester.

### (1-Oxy-benzo[1,2,4]triazin-3-yl)-(6-piperazin-1-yl-pyridin-3-yl)-amine

To (1-Oxy-benzo[1,2,4]triazin-3-yl)-(6-piperazin-1-yl-pyridin-3-yl)-amine (0.45 g) in DCM (30 ml) was slowly added TFA (4.5 ml) over a period of 10 min. The reaction mixture was allowed to stir for a further 45 minutes at RT. On completion, water (200 ml) was added to the reaction mixture and the organic layer was separated and the aqueous layer back extracted with ethyl acetate (100 ml). The aqueous layer was basified with sodium carbonate solution to pH˜8. The resulting solid was filtered and dried to give the title compounds.

### Example 21

### (6-Chloro-benzo[1,2,4]triazin-3-yl)-pyridin-3-yl-amine

Ammonium formate (0.13 g) was added to (6-Chloro-1-oxy-benzo[1,2,4]triazin-3-yl)-pyridin-3-yl-amine (0.2 g) and zinc dust (0.09 g) in methanol (30 ml). The reaction mixture was then stirred at 70° C. for 10 hrs. On completion, the reaction mixture was cooled to RT and filtered through a celite bed. The methanol was removed under reduced pressure to give a residue, to which water (˜100 ml) was added. The reaction mixture was extracted with ethyl acetate (4×100 ml). The combined organic extracts were dried over sodium sulfate and the solvent was removed under reduced pressure to give a residue, which was further purified by column chromatography (SiO₂, 20% EtOAc/n-hexane) yielding the title compound.

### Example 22

### (6-Chloro-1-oxy-benzo[1,2,4]triazin-3-yl)-pyridin-3-yl-amine

The title compound was prepared following the procedure outlined in example 23.

### Examples 23

### 4-Nitro-biphenyl-3-ylamine

5-Chloro-2-nitro-phenylamine (9 g), Phenyl boronic acid (9.5 g), BINAP (1.62 g), potassium carbonate (18 g) and 1,4-Dioxane (270 ml) were heated to 90° C. and the reaction mixture was stirred at for 24 hrs. On completion, the reaction mixture was filtered through a celite bed. The solvent was removed under reduced pressure to give a residue, which was further purified by column chromatography (SiO₂, 5% EtOAc/n-hexane)

### 1-Oxy-6-phenyl-benzo[1,2,4]triazin-3-ylamine

4-Nitro-biphenyl-3-ylamine (4 g) and cyanamide (7.8 g) were heated for 30 min at 100° C. The reaction mixture was cooled to 50° C. and conc. HCl (40 ml) was added slowly over a period of 30 min. The reaction mixture was again heated to 100° C. for 2 h. Cyanamide (7.8 g) was added to the above reaction mixture at 100° C. and stirred for 4 h. The reaction mixture was cooled to 20° C. and 30% NaOH sol. (340 ml) was added slowly at 20° C. The reaction mixture was again heated to 100° C. for 1½ h. The reaction mixture was cooled to RT and the resulting solid was filtered. The solid was washed with water (2×50 ml), ether (2×50 ml) and dried to give 1-Oxy-6-phenyl-benzo[1,2,4]triazin-3-ylamine.

### 1-Oxy-6-phenyl-benzo[1,2,4]triazin-3-ol

Concentrated H₂SO₄ (1.92 ml) was slowly added to a solution of 1-Oxy-6-phenyl-benzo[1,2,4]triazin-3-ylamine (0.96 g) in CH₃COOH: water (19.2 ml). The reaction mixture was stirred at 100° C. for 30 min and then cooled to 50° C. NaNO₂ (0.67 g) in water (2 ml) was added dropwise to the reaction mixture over a period of 10 min at 50° C. The reaction was heated to 100° C. for 1 h. On completion, the mixture was cooled to RT and kept in refrigerator overnight. The resulting solid was filtered, washed with water (2×50 ml) and dried.

### 3-Chloro-6-phenyl-benzo[1,2,4]triazine 1-oxide

1-Oxy-6-phenyl-benzo[1,2,4]triazin-3-ol (0.58 g) in POCl₃ (11.6 ml) was stirred at 100° C. for 2 hrs with a catalytic amount of N,N-Dimethylaniline. On completion, excess POCl₃ was removed under reduced pressure and the resulting residue was poured on to an ice water mixture. The resulting solid was filtered, washed with water (20 ml) and dried to give 3-Chloro-6-phenyl-benzo[1,2,4]triazine 1-oxide.

### (1-Oxy-6-phenyl-benzo[1,2,4]triazin-3-yl)-pyridin-3-yl-amine

To a mixture of 3-Chloro-6-phenyl-benzo[1,2,4]triazine 1-oxide (0.5 g), 3-Amino pyridine (0.3 g) and DMSO (10 ml) were taken in the RB flask to which 60% NaH (0.13 g) was added. The reaction mixture was heated to 70-80° C. for 1½ h. On completion, the reaction mixture was cooled to RT and water (˜50 ml) was added. The reaction mixture was extracted with ethyl acetate (4×150 ml). The combined organic extracts were dried over sodium sulfate. The solvent was removed under reduced pressure to give a residue, which was further purified by column chromatography (SiO₂, 40% EtOAc/n-hexane) yielding (1-Oxy-6-phenyl-benzo[1,2,4]triazin-3-yl)-pyridin-3-yl-amine.

### (6-Phenyl-benzo[1,2,4]triazin-3-yl)-pyridin-3-yl-amine

(1-Oxy-6-phenyl-benzo[1,2,4]triazin-3-yl)-pyridin-3-yl-amine (0.16 g), sodium dithionite (0.16 g) and acetic acid (24 ml) were heated to 100° C. for 1 h. The remaining sodium dithionite (0.16 g) was added and heating continued at 100° C. for a further 1½ hrs. On completion, the reaction mixture was cooled to RT and the acetic acid was removed under reduced pressure. The reaction mixture was basified with sat. sodium bicarbonate solution (˜10 ml) to attain pH˜8. The mixture was extracted with ethyl acetate (3×100 ml) and the combined organic extracts were dried over sodium sulfate. The solvent was removed under reduced pressure to give a residue, which was further purified by column chromatography (SiO₂, 40% EtOAc/n-hexane) yielding (6-Phenyl-benzo[1,2,4]triazin-3-yl)-pyridin-3-yl-amine.

### Examples 24

### (1-Oxy-6-phenyl-benzo[1,2,4]triazin-3-yl)-pyridin-3-yl-amine

The title compound was prepared as described in example 23.

### Example 25

### 1-Oxy-6-piperazin-1-yl-benzo[1,2,4]triazin-3-ylamine

6-Fluoro-1-oxy-benzo[1,2,4]triazin-3-ylamine (2.78 mmoles) was dissolved in 50 ml DMF and 40% of piperidine in water was added (10 equivalents). The reaction mixture was stirred at 40° C. for 2 hours after which the reaction was cooled to room temperature and the precipitate filtered and washed with THF and ether to yield 0.527 g of the desired product.

### Example 26

### 6-Piperazin-1-yl-benzo[1,2,4]triazin-3-ylamine

1-Oxy-6-piperazin-1-yl-benzo[1,2,4]triazin-3-ylamine (1.2 mmoles) was suspended in 20 mls of 70% ethanol and 1.20 equivalents of Na₂S₂O₄ were added. The reaction mixture was refluxed overnight after which the reaction was cooled to room temperature and the precipitate filtered and washed with ethanol. Diethyl ether was added to the resulting filtrate and the product was filtered, washed with diethyl ether and dried.

### Example 27

### 4-(3-Fluoro-4-nitro-phenoxymethyl)-piperidine-1-carboxylic acid tert-butyl ester

3-Fluoro-4-nitro-phenol (23 mmoles), 4-Hydroxymethyl-piperidine-1-carboxylic acid tert-butyl ester (23 mmoles) and triphenylphosphine (23 mmoles) were mixed together in 50 ml of tetrahydrofuran (THF) and Diisopropyl azodicarboxylate (DIAD—23 mmoles) was added slowly. The reaction mixture was stirred for 1 hour. The crude is purified by column chromatography eluting 20-40% ethyl acetate/hexanes.

### 4-(3-Amino-1-oxy-benzo[1,2,4]triazin-6-yloxymethyl)-piperidine-1-carboxylic acid tert-butyl ester

4-(3-Fluoro-4-nitro-phenoxymethyl)-piperidine-1-carboxylic acid tert-butyl ester (10 mmol) was dissolved in 50 ml of THF and 84 mmoles of guanidine (prepared by mixing guanidine hydrochloride and 1.0 eq of potassium tert-butoxide in Ethanol, removal of the salt precipitate and evaporation of the ethanol) was added and the mixture stirred at reflux overnight. The reaction is cooled and the upper layer decanted and washed 3 times with THF (50 ml×3). The combined THF solution was concentrated and purified by column chromatography eluting 10% MeOH/Ethyl acetate and hexanes.

### 1-Oxy-6-(piperidin-4-ylmethoxy)-benzo[1,2,4]triazin-3-ylamine.hydrochloride

4-(3-Amino-1-oxy-benzo[1,2,4]triazin-6-yloxymethyl)-piperidine-1-carboxylic acid tert-butyl ester (0.5 g) was dissolved in 10 ml DCM and 4N HCl in dioxane was added. The precipitate is filtered and washed with ether to give the desired product.

### Example 28

### 4-(3-Amino-benzo[1,2,4]triazin-6-yloxymethyl)-piperidine-1-carboxylic acid tert-butyl ester

4-(3-Amino-1-oxy-benzo[1,2,4]triazin-6-yloxymethyl)-piperidine-1-carboxylic acid tert-butyl ester (1.0 g) was mixed with 2.0 equivalents of Na₂S₂O₄ in 15 mls of 70% ethanol and the reaction mixture refluxed for 1 hour. The product was purified by column chromatography eluting 50% ethyl acetate/hexanes.

### 6-(Piperidin-4-ylmethoxy)-benzo[1,2,4]triazin-3-ylamine.hydrochloride

4-(3-Amino-benzo[1,2,4]triazin-6-yloxymethyl)-piperidine-1-carboxylic acid tert-butyl ester (0.4 g) was dissolved in 5 mls of DCM to which 2 mls of 4N HCl in dioxane was added. The reaction mixture was stirred at room temperature overnight after which the product was filtered and washed with ether.

### Example 29

### 2-Fluoro-4-[2-(3,4-Dichloro-phenyl)-ethoxy]-1-nitro-benzene

3-Fluoro-4-nitro-phenol (10 mmoles), 2-(3,4-Dichloro-phenyl)-ethanol (10 mmoles) and triphenylphosphine (10 mmoles) were mixed together in 50 mls of THF and DIAD (10 mmoles) was added slowly. The reaction mixture was stirred for 1 hour following which the crude product was purified by column chromatography eluting 40% ethyl acetate/hexanes.

### 6-[2-(3,4-Dichloro-phenyl)-ethoxy]-1-oxy-benzo[1,2,4]triazin-3-ylamine

2-Fluoro-4-[2-(3,4-Dichloro-phenyl)-ethoxy]-1-nitro-benzene (2.5 g) was dissolved in 50 mls of THF and 84 mmoles of guanidine (prepared by mixing guanidine hydrochloride and 1.0 equivalent of potassium tert-butoxide) was added. The reaction mixture was stirred at 70° C. overnight following which, the reaction was cooled to room temperature and diluted with water. The resulting precipitate was filtered and washed with water and ethanol. This material was further heated in 20 mls of ethanol to reflux and cooled to room temperature. The precipitate was filtered and washed with ether and dried the give the desired product.

### Example 30

### 6-[2-(3,4-Dichloro-phenyl)-ethoxy]-benzo[1,2,4]triazin-3-ylamine

6-[2-(3,4-Dichloro-phenyl)-ethoxy]-1-oxy-benzo[1,2,4]triazin-3-ylamine (0.86 g) was mixed with 1.5 equivalent of Na₂S₂O₄ in 15 mls of 70% ethanol and refluxed for 3 days. The reaction is cooled to room temperature and the product was filtered and washed with water, ethanol and ether.

### Example 31

### 1-Benzo[1,2,4]triazin-6-yl-3-pyrazin-2-yl-urea

Compound described in example 18

### Example 32

### 1-(3-Amino-benzo[1,2,4]triazin-6-yl)-3-pyrazin-2-yl-urea

Compound described in example 18

The resulting products of Examples 1-32 were submitted to analysis by Nuclear Magnetic Resonance Spectroscopy (NMR), Liquid Chromatography and Mass Spectroscopy (MS)

The results are set forth in Table 4.

TABLE 4

| Ex | | | LCMS | MS |
|---|---|---|---|---|
| Num | Name | ¹H NMR (DMSO d6) | RT | (ES+) |
| 1 | 1-Oxy- | δ8.14 (dd, 1H), 7.78 (t, 1H), | 1.40 | 163.2 |
| | benzo[1,2,4]triazin-3- | 7.54 (d, 1H), 7.34 (br s, 1H), | | |
| | ylamine | 7.346 (t, 1H) | | |
| 2 | Benzo[1,2,4]triazin-3- | δ8.25 (d, 1H), 7.86 (t, 1H), | 1.28 | 147 |
| | ylamine | 7.70 (Br s, 2H), 7.59 (d, 1H), | | |
| | | 7.52 (t, 1H) | | |
| 3 | Benzo[1,2,4]triazin-3- | δ9.18 (d, 1H), 8.48 (dt, 1H), | 2.18 | 224.3 |
| | yl-pyridin-3-yl-amine | 8.43 (d, 1H), 8.34 (d, 1H), | | |
| | | 8.03 (t, 1H), 7.88 (d, 1H), | | |
| | | 7.73 (t, 1H) 7.49 (dd, 1H) | | |
| 4 | (1-Oxy- | δ10.54 (s, 1H), 9.08 (s, 1H), | 2.17 | 240.3 |
| | benzo[1,2,4]triazin-3- | 8.40 (d, 1H), 8.34 (br s, 1H), | | |
| | yl)-pyridin-3-yl-amine | 8.32 (d, 1H), 7.99 (t, 1H), | | |
| | | 7.87 (d, 1H), 7.59 (t, 1H) | | |
| | | 7.49 (dd, 1H) | | |
| 5 | (1-Oxy- | δ10.05 (s, 1H), 8.26 (d, 1H), | 2.44 | 323 |
| | benzo[1,2,4]triazin-3- | 7.88-7.85 (m, 1H), 7.69 (d, | | |
| | yl)-(4-piperazin-1-yl | 3H), 7.46-7.42 (m, 1H), | | |
| | phenyl)-amine | 6.96 (d, 1H), 3.05 (br s, 4H), | | |
| | | 2.89 (br s, 1H) | | |
| 6 | Benzo[1,2,4]triazin-3- | δ10.74 (s, 1H), | 2.46 | 307 |
| | yl-(4-piperazin-1-yl- | 8.36-8.31 (m, 2H), 7.97-7.93 (m, 1H), | | |
| | phenyl)-amine | 7.88 (d, 2H), 7.75 (d, 1H), | | |
| | | 7.65-7.61 (m, 1H), 7.06) d, | | |
| | | 2H), 3.2 (br s, 4H), 3.07 (br | | |
| | | s, 1H) | | |
| 7 | 1-Oxy-7-(1H-pyrazol-4- | δ8.51 (br s, 1H), 8.37 (d, | 1.43 | 229.4 |
| | yl)-benzo[1,2,4]triazin- | 2H), 7.63 (d, 2H), 7.36 (s, | | |
| | 3-ylamine | 2H) | | |
| 8 | 7-(1H-Pyrazol-4-yl)- | δ8.49 (br s, 2H), 8.20 (d, | 1.39 | 213.3 |
| | benzo[1,2,4]triazin-3- | 2H), 7.62 (d, 3H) | | |
| | ylamine | | | |
| 9 | 1-Benzo[1,2,4]triazin-3- | δ8.60 (d, 1H), 8.45 (d, 1H), | 3.35 | 401.4 |
| | yl-3-[5-chloro-2-(3- | 8.25-8.20 (m, 1H), 8.00 (d, | | |
| | dimethylamino- | 1H), 7.27 (d, 1H), 7.22 (dd, | | |
| | propoxy)-phenyl]-urea | 1H), 4.39 (d, 2H), 3.44 (br s, | | |
| | | 6H), 2.60 (s, 6H), | | |
| | | 2.50-2.42 (m, 2H), 2.25-2.20 (m, 2H) | | |
| 10 | (5-Methyl-1H-pyrazol- | δ8.38 (d, 1H), 8.16 (d, 1H), | 1.78 | 243.3 |
| | 3-yl)-(1-oxy- | 8.09 (t, 1H), 7.77 (t, 1H), | | |
| | benzo[1,2,4]triazin-3- | 6.91 (s, 2H), 5.37 (s, 1H), | | |
| | yl)-amine | 2.15 (s, 3H) | | |
| 11 | Benzo[1,2,4]triazin-3- | δ8.61 (d, 1H), 8.22-8.20 (m, | 1.64 | 227 |
| | yl-(5-methyl-1H- | 2H), 8.00-7.96 (m, 1H), | | |
| | pyrazol-3-yl)-amine | 6.91 (s, 2H), 5.44 (s, 1H), 2.22 (s, | | |
| | | 3H) | | |
| 12 | 3-(1H-Pyrazol-4-yl)- | δ8.47-8.44 (m, 3H), | 1.82 | 214.3 |
| | benzo[1,2,4]triazine 1- | 8.14-8.10 (m, 2H), 7.88-7.84 (m, | | |
| | oxide | 1H) | | |
| 13 | 3-(1H-Pyrazol-4-yl)- | δ8.84 (br s, 1H), 8.59 (d, | 1.75 | 198.3 |
| | benzo[1,2,4]triazine | 1H), 8.46 (br s, 1H), | | |
| | | 8.21-8.12 (m, 2H), 8.02-7.98 (m, | | |
| | | 1H) | | |
| 14 | 7-(2H-Pyrazol-3-yl)- | δ8.58 (br s, 1H), 8.37 (d, | 1.52 | 213.3 |
| | benzo[1,2,4]triazin-3- | 1H), 7.90 (s, 1H), 7.65 (br s, | | |
| | ylamine | 2H), 7.61 (d, 1H), 6.99 (br s, | | |
| | | 1H) | | |
| 15 | 1-Oxy-7-(2H-pyrazol-3- | δ8.60 (br s, 1H), 8.41 (d, | 1.55 | 229.3 |
| | yl)-benzo[1,2,4]triazin- | 1H), 7.97 (s, 1H), 7.69 (d, | | |
| | 3-ylamine | 1H), 7.48 (br s, 2H), 7.02 (s, | | |
| | | 1H) | | |
| 16 | 3-(1H-Pyrazol-3-yl)- | δ8.61 (d, 1H), 8.19 (br s, | 1.71 | 198.3 |
| | benzo[1,2,4]triazine | 2H), 8.04 (br s, 1H), | | |
| | | 7.80-7.76 (m, 1H), 7.30 (s, 1H) | | |
| 17 | 3-(1H-Pyrazol-3-yl)- | δ8.49 (d, 1H), 8.20-8.18 (m, | 1.75 | 214.3 |
| | benzo[1,2,4]triazine 1- | 2H), 8.06-7.90 (m, 2H), | | |
| | oxide | 7.14 (s, 1H) | | |
| 18 | 1-Benzo[1,2,4]triazin-6- | δ10.46 (br s, 1H), 9.92 (br s, | 1.63 | 2.68 |
| | yl-3-pyrazin-2-yl-urea | 1H), 9.88 (s, 1H), 9.14 (d, | | |
| | | 1H), 8.49 (d, 1H), 8.40 (br s, | | |
| | | 2H), 8.35 (s, 1H) 7.94 (dd, | | |
| | | 1H) | | |
| 19 | Benzo[1,2,4]triazin-3- | δ10.63 (s, 1H), 8.68 (s, 1H), | 2.16 | 308.4 |
| | yl-(6-piperazin-1-yl- | 8.29 (d, 1H), 8.02 (d, 1H), | | |
| | pyridin-3-yl)-amine | 7.89 (t, 1H), 7.69 (d, 1H), | | |
| | | 7.57 (t, 1H), 6.88 (d, 1H) | | |
| | | 3.37-3.35 (m, 4H), | | |
| | | 2.80-2.78 (m, 4H) | | |
| 20 | (1-Oxy- | δ9.97 (s, 1H), 8.54 (s, 1H), | 2.35 | 324.4 |
| | benzo[1,2,4]triazin-3- | 8.19 (d, 1H), 7.91 (d, 1H), | | |
| | yl)-(6-piperazin-1-yl- | 7.85 (t, 1H), 7.67 (d, 1H), | | |
| | pyridin-3-yl)-amine | 7.43 (t, 1H), 6.85 (d, 1H), | | |
| | | 3.37-3.32 (m, 4H), | | |
| | | 2.80-2.78 (m, 4H) | | |
| 21 | (6-Chloro- | δ11.23 (s, 1H), 9.11 (d, 1H), | 2.66 | 258.3 |
| | benzo[1,2,4]triazin-3- | 8.44-8.41 (m, 1H), 8.40 (d, | | |
| | yl)-pyridin-3-yl-amine | 1H), 8.31 (dd, 1H), 7.96 (d, | | |
| | | 1H), 7.67 (dd, 1H) 7.44 (dd, | | |
| | | 1H) | | |
| 22 | (6-Chloro-1-oxy- | δ10.57 (s, 1H), 8.99 (d, 1H), | 2.66 | 274.3 |
| | benzo[1,2,4]triazin-3- | 8.34-8.31 (m, 1H), 8.29 (d, | | |
| | yl)-pyridin-3-yl-amine | 1H), 8.24 (d, 1H), 7.93 (s, | | |
| | | 1H), 7.51 (d, 1H) 7.41 (dd, | | |
| | | 1H) | | |
| 23 | (6-Phenyl- | δ11.11 (s, 1H), 9.16 (d, 1H), | 3.12 | 300.4 |
| | benzo[1,2,4]triazin-3- | 8.50-8.47 (m, 1H), 8.44 (d, | | |
| | yl)-pyridin-3-yl-amine | 1H), 8.29 (dd, 1H), 8.09 (d, | | |
| | | 1H), 8.03 (dd, 1H) 7.97 (d, | | |
| | | 2H), 7.58 (t, 2H), 7.52 (d, | | |
| | | 1H), 7.44 (dd, 1H) | | |
| 24 | (1-Oxy-6-phenyl- | δ10.47 (s, 1H), 9.05 (d, 1H), | 3.13 | 316.3 |
| | benzo[1,2,4]triazin-3- | 8.40-8.37 (m, 1H), 8.32 (d, | | |
| | yl)-pyridin-3-yl-amine | 1H), 8.28 (dd, 1H), 8.07 (d, | | |
| | | 1H), 7.91 (d, 2H) 7.87 (d, | | |
| | | 1H), 7.59-7.49 (m, 3H), | | |
| | | 7.42 (dd, 1H) | | |
| 25 | 1-Oxy-6-piperazin-1-yl- | δ7.90 (d, 1H), 7.16 (dd, 1H), | 1.29 | 247.3 |
| | benzo[1,2,4]triazin-3- | 6.99 (s, 2H), 6.55 (d, 1H), | | |
| | ylamine | 3.36-3.32 (m, 4H), | | |
| | | 2.82-2.79 (m, 4H) | | |
| 26 | 6-Piperazin-1-yl- | δ7.91 (d, 1H), 7.33 (dd, 1H), | 1.20 | 231.3 |
| | benzo[1,2,4]triazin-3- | 7.17 (s, 2H), 6.59 (d, 1H), | | |
| | ylamine | 3.58-3.56 (m, 4H), | | |
| | | 3.11-3.08 (m, 4H) | | |
| 27 | 1-Oxy-6-(piperidin-4- | δ9.11-9.09 (br m, 1H), | 1.72 | 276.3 |
| | ylmethoxy)- | 8.78-8.75 (br m, 1H), 8.05 (d, | | |
| | benzo[1,2,4]triazin-3- | 1H), 6.96-6.90 (m, 2H), | | |
| | ylamine•hydrochloride | 4.03 (d, 2H), 3.29 (d, 2H), | | |
| | | 2.90 (q, 2H), 2.15-2.07 (br s, 1H), | | |
| | | 1.92 (d, 2H), 1.57-1.47 (m, | | |
| | | 2H) | | |
| 28 | 6-(Piperidin-4- | δ9.00-8.97 (br m, 1H), | 1.58 | 260.3 |
| | ylmethoxy)- | 8.71-8.60 (br m, 1H), 8.05 (d, | | |
| | benzo[1,2,4]triazin-3- | 1H), 7.07 (dd, 1H), 6.85 (d, | | |
| | ylamine•hydrochloride | 1H), 4.05 (d, 2H), 3.30 (d, | | |
| | | 2H), 2.91 (q, 2H), | | |
| | | 2.17-2.08 (br s, 1H), 1.93 (d, 2H), | | |
| | | 1.57-1.47 (m, 2H) | | |
| 29 | 6-[2-(3,4-Dichloro- | δ8.02 (d, 1H), 7.67 (s, 1H), | 3.33 | 351.2 |
| | phenyl)-ethoxy]-1-oxy | 7.59 (d, 1H), 7.36 (dd, 1H), | | |
| | benzo[1,2,4]triazin-3- | 7.28 (s, 2H), 6.92-6.89 (m, | | |
| | ylamine | 2H), 4.37 (t, 2H), 3.10 (t, | | |
| | | 2H) | | |
| 30 | 6-[2-(3,4-Dichloro- | δ8.02 (d, 1H), 7.67 (s, 1H), | 3.25 | 335.3 |
| | phenyl)-ethoxy]- | 7.59 (d, 1H), 7.45 (br s, 2H), | | |
| | benzo[1,2,4]triazin-3- | 7.37 (d, 1H), 7.04 (dd, 1H), | | |
| | ylamine | 6.85 (d, 1H), 4.39 (t, 2H), | | |
| | | 3.12 (t, 2H) | | |
| 31 | 1-(3-Amino-1-oxy- | δ12.17 (s, 1H), 10.89 (s, | 1.70 | 299.3 |
| | benzo[1,2,4]triazin-6- | 1H), 9.38 (s, 1H), | | |
| | yl)-3-pyrazin-2-yl-urea | 8.42-8.39 (m, 2H), 7.98 (d, 1H), | | |
| | | 7.35 (br s, 2H), 7.19 (dd, 1H) | | |
| | | 6.56 (br s, 1H) | | |
| 32 | 1-(3-Amino- | δ10.07 (s, 1H), 9.73 (s, 1H), | 1.69 | 283.3 |
| | benzo[1,2,4]triazin-6- | 9.11 (s, 1H), 8.38-8.37 (m, | | |
| | yl)-3-pyrazin-2-yl-urea | 1H), 8.32 (d, 1H), 8.09 (d, | | |
| | | 1H), 7.83 (d, 1H), | | |
| | | 7.31-7.30 (m, 3H) | | |

| | |
|---|---|
| LCMS/MS: | Waters 2795, Micromass ZQ, 996 Diode array (220-400) |
| Method: | Flow 2 ml/min. Run time 5.8 min. Gradient: 95% Aq to 5% |
| | over 4.25 min. 5% Aq from 4.25-5.8 min |
| | Aq: 10 mM Ammonium formate. Org: 95% |
| | Acetonitirle/0.1% Ammonia/5% Aq |
| Column: | phenomenex 5 micron C18 30 × 4.6 mm |
| NMR: | Bruker 400 MHz |

### Example 33

### Biological Assays For Kinase Inhibition

The compounds of the invention prepared in Examples 1-32 were tested for kinase inhibition against a range of common protein kinases. All kinase assays were conducted at Upstate Ltd, Gemini Crescent, Dundee Technology Park, Dundee, DD2 1SW, UK in accordance with its standard protocols as follows:

### Arg (h)

In a final reaction volume of 25 μl, Arg (h) (5-10 mU) is incubated with 8 mM MOPS pH 7.0, 0.2 mM EDTA, 50 μM EAIYAAPFAKKK, 10 mM MgAcetate and [□-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### Abl (h)

In a final reaction volume of 25 μl, Abl (h) (5-10 mU) is incubated with 8 mM MOPS pH 7.0, 0.2 mM EDTA, 50 μM EAIYAAPFAKKK, 10 mM MgAcetate and [□-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### Aurora-A (h)

In a final reaction volume of 25 μl, Aurora-A (h) (5-10 mU) is incubated with 8 mM MOPS pH 7.0, 0.2 mM EDTA, 200 μM LRRASLG (Kemptide), 10 mM MgAcetate and □□-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

### CHK1 (h)

In a final reaction volume of 25 μl, CHK1 (h) (5-10 mU) is incubated with 8 mM MOPS pH 7.0, 0.2 mM EDTA, 200 μM KKKVSRSGLYRSPSMPENLNRPR, 10 mM MgAcetate and □□-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### KDR (h)

In a final reaction volume of 25 μl, KDR (h) (5-10 mU) is incubated with 8 mM MOPS pH 7.0, 0.2 mM EDTA, 0.33 mg/ml myelin basic protein, 10 mM MgAcetate and [□-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

p70S6K (h)

In a final reaction volume of 25 μl, p70S6K (h) (5-10 mU) is incubated with 8 mM MOPS pH 7.0, 0.2 mM EDTA, 100 μM KKRNRTLTV, 10 mM MgAcetate and [□-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

The results of these assays are summarized below in Table 5. As can be seen from these results, many of the compounds of the invention are effective inhibitors of one or more of these common protein kinases.

Data for compounds. ATP concentrations are: Arg=45 uM; abl=45 uM; AuroraA=15 uM; Chk1=90 uM; KDR=90 uM; P70S6=15 uM. Figures represent the % inhibition when tested at 100 uM of inhibitor.

TABLE 5

| Ex | Arg | Abl | AuroraA | | KDR | |
|---|---|---|---|---|---|---|
| Num | (h) | (h) | (h) | Chk1 (h) | (h) | P70S6 (h) |
| 3 | 71 | 65 | 68 | 21 | | 9 |
| 4 | | | 64 | 14 | | 0 |
| 5 | 78 | 83 | 24 | 7 | | |
| 6 | 92 | 89 | 41 | 33 | | |
| 7 | 81 | 86 | 98 | 31 | 98 | 99 |
| 8 | 82 | 87 | 100 | 26 | 96 | 97 |
| 9 | | | | 93 | | |
| 10 | | | 40 | 20 | | |
| 11 | 40 | 36 | 37 | 32 | | |
| 12 | | | 69 | 3 | 97 | 87 |
| 13 | | | 90 | 13 | 80 | 92 |
| 14 | | | 81 | 20 | 89 | 47 |
| 15 | | | 90 | 10 | 46 | 29 |
| 16 | | | 41 | | 17 | 10 |
| 17 | | | 39 | | 0 | 0 |
| 18 | | | | 23 | | |
| 19 | 95 | 89 | 79 | | | |
| 20 | 77 | 72 | 52 | 1 | | |
| 21 | 84 | 76 | 58 | | | |
| 22 | | | 82 | | | |
| 23 | 83 | 80 | 68 | | | |
| 24 | | | 92 | | | |
| 25 | | | | | 18 | 43 |
| 26 | | | | | 98 | 37 |
| 27 | | | | | 3 | 62 |
| 28 | | | | | 6 | 47 |
| 29 | | | | | 0 | 49 |
| 30 | | | | | 11 | 52 |

### Example 34

### Measurement of the One Election Reductio Potentials of the Compounds of the Invention

The one electron reduction potential of the compounds of the invention may be measured by techniques known in the art. Drugs that are candidates for hypoxia-selective action via reduction to a free-radical intermediate reactive towards oxygen are typically nitroimidazoles, nitrobenzenes, quinones, and aromatic N-oxides. They are ‘bioreductively’ activated by reductase enzymes to a free-radical intermediate by the addition of a single electron from the enzyme (usually a flavoprotein) to the drug D:

D+electron→D.⁻ (drug radical)

If the electron affinity of the drug is less than that of oxygen, then in well-oxygenated cells or tissue, oxygen reverses reduction:

D.⁻+O₂→D+O₂.⁻.

The drug is restored but superoxide radicals are formed. However, the cell has antioxidant defences against it. In anoxic or hypoxic tissues the drug radical can go on to form further reduction products which may bind to cellular materials or be otherwise toxic.

The electron affinity is measured by the reduction potential, which in this case is that for addition of one electron, represented by the symbol E qualified by the ‘couple’ involved (oxidant/reductant), e.g. E(D/D.⁻). Sometimes the pH may be subscripted: E₇, or E may be described as a mid-point potential (Eₘ), or a one-electron potential (E¹₇).

If the free radical D.⁻ is stable at the pH of interest, conventional electrochemical techniques may be used to measure E(D/D.⁻). In most cases, however, the radical is short-lived. In this case the relative electronic affinity (relative to a redox indicator (Ind)) may be quantified by measuring the position of the electron-transfer equilibrium:

D.⁻+Ind⇄D+Ind.⁻.

If the equilibrium is over to the right (equilibrium constant K>1), then Ind is more electronic-affinic than D (i.e. E(Ind/Ind.⁻) is more positive than E(D/D.⁻). If over to the left (K<1), the reverse. The equilibrium constant is defined as:

K=([D] [Ind.⁻])/([D.⁻] [Ind])

where square brackets denote concentrations. The relationship between K and the reduction potential difference ΔE is:

ΔE=E(Ind/Ind.⁻)−E(D/D.⁻)=0.05915 log K

if E is in volts. (Typical values of useful bioreductive’ drugs are around −0.3 to −0.6 V since the value of E(O₂/O₂.⁻) expressed on the same scale is close to −0.2 V, measured against a hydrogen electrode standard, NHE, and cellular enzymes reduce drugs of lower potential very slowly.) If the value E(Ind/Ind.⁻) is known (e.g. for some viologens such as paraquat where the radical is stable), then the value for E(D/D.⁻) is calculable.

The challenge experimentally is to generate the electron-transfer equilibrium between D and Ind, by generating D.⁻ and/or Ind.⁻ in a solution containing both D and Ind, and measure K before any of the radicals can decay via other routes. Ideally, the approach to equilibrium should be observed so that one is confident that the desired chemistry is occurring. This also provides a second, independent route to calculate K. If low concentrations of radicals are formed such that [D.⁻] and [Ind.⁻] are both much less than [D] and [Ind], then the kinetics of the approach to equilibrium are exponential and characterized by a (first-order) rate constant k_{obs}, measured in s⁻¹. Then:

k_{obs}=k_{f}[Ind]+kᵣ[D]

where k_{f}, kᵣ are the rate constants for the forward and reverse reaction; a linear plot of k_{obs}/[D] vs. [Ind]/[D] has a slope k_{f} and intercept kᵣ. Then K=k_{f}/kᵣ.

A method known as ‘pulse radiolysis’ is a powerful technique in which the desired radicals can be produced in about a microsecond and the equilibrium position of the reaction between D.⁻ and Ind measured in a few tens of microseconds, typically. The radicals produced by irradiating water with a sub-microsecond pulse of high-energy electrons (typically 1-10 MeV) are both oxidizing and reducing but the former (.OH) can be transformed to the latter by adding an alcohol such as 2-propanol, or formate. These secondary radicals then reduce D and/or Ind to D.⁻ and/or Ind.⁻:

H₂O→irradiate→e_{aq}⁻+.OH+H.

e_{aq}⁻+D (or Ind)→D.⁻ (or Ind.⁻)

.OH (or H.)+(CH₃)₂CHOH→H₂O (or H₂)+(CH₃)C.OH

(CH₃)₂C.OH+D (or Ind)→(CH₃)₂CO+H⁺+D.⁻ (or Ind.⁻)

.OH (or H.)+HCO₂⁻→H₂O (or H₂)+CO₂.⁻

CO₂.⁻+D (or Ind)→CO₂+D.⁻ (or Ind.⁻).

The concentrations of radicals are measured by spectrophotometry. Typically, a display of light absorbance vs. time (over microseconds to milliseconds) is obtained, at wavelengths where either D.⁻ or Ind.⁻ absorb (or both), from which concentrations of radicals are calculated. Radical concentrations are usually a few micromolar whereas concentrations of D and Ind are typically 20 micromolar to 2 millimolar so that D and Ind are little depleted. Concentrations of 2-propanol or formate are typically 0.1 molar such that unwanted reactions between .OH and H. and D or Ind are avoided.

One of the earliest examples of measuring these relative electron affinities using pulse radiolysis was described by Patel and Willson (K B Patel and R L Willson. Semiquinone free radicals and oxygen. Pulse radiolysis study of one electron transfer equilibria. Journal of the Chemical Society, Faraday Transactions 1, 1973, 69, 814-825). This was applied to nitroaromatics and quinones, e.g. D Meisel and P Neta. One-electron redox potentials of nitro compounds and radiosensitizers. Correlation with spin densities of their radical anions. Journal of the American Chemical Society, 1975, 97, 5198-5203; P Wardman and E D Clarke. One-electron reduction potentials of substituted nitroimidazoles measured by pulse radiolysis. Journal of the Chemical Society, Faraday Transactions 1, 1976, 72, 1377-1390. A review describes many further examples and some refinements of the mathematical treatment: P Wardman. Reduction potentials of one-electron couples involving free radicals in aqueous solution. Journal of Physical and Chemical Reference Data, 1989, 18, 1637-1755.

What is claimed is:

1. A method of inhibiting protein kinase activity associated with tumor growth, cell proliferation or angiogenesis, comprising administering to a mammal in need thereof a therapeutically effective amount of a heterocyclic triazine compound of the Formula I: 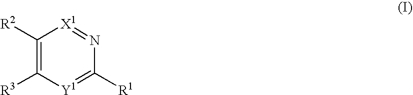
Wherein:
both X¹ and Y¹ are N-oxide; or
one of X¹ and Y¹ is N and the other is N or N-oxide;
R¹ is H, OH, NH₂, NHR⁴, a 5- to 7-membered heterocyclic ring which is unsaturated and which may contain one or more heteroatoms selected from O, N and S and which is unsubstituted or substituted on any ring carbon or ring heteroatom, or an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system;
R² and R³ are each independently selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶ )CON(R⁷)(R⁸), —N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸)
wherein A is a C₁-C₆ alkylidine group that is optionally interrupted by —O—, —S—, C(O)— or —N(R⁶), and wherein
R² and R³ may form, together with the carbon atoms to which they are attached, a fused benzene ring or a fused 5- to 7-membered heterocyclic ring which is saturated or unsaturated and which may contain one or more heteroatoms selected from O, N and S, the benzene ring or heterocyclic ring being unsubstituted or substituted;
wherein R⁶ is H, C₁-C₆ alkyl which is unsubstituted or substituted, C₃-C₁₀ cycloalkyl which is unsubstituted or substituted or a 5- to 7-membered heterocyclic ring which is unsaturated or saturated and which contains one or more heteroatoms selected from O, N and S, and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different, and wherein
R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;
p is 0 or an integer from 1 to 5;
q is an integer from 1 to 6;
R⁴ is H, C₁-C₆ alkyl which is unsubstituted or substituted, C₃-C₁₀ cycloalkyl which is unsubstituted or substituted, aryl which is unsubstituted or substituted, a 5- to 7-membered heterocyclic group which is unsaturated or saturated, which contains one or more heteroatoms selected from O, N and S and which is unsubstituted or substituted on any ring carbon or ring heteroatom, the carbocyclic group or heterocyclic group R⁴ being optionally substituted by one or more substituent selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(⁷)(R⁸), —N(R⁶)C(O)R⁶, —N(R⁶)COOR⁶, —N(R⁶)CON(R⁶)₂, —N(R⁶ )CON(R⁷)(R⁸)—N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸) wherein A is a C₁-C₆ alkylidine group that is optionally interrupted by —O—, —S—, —C(O)— or —N(R⁶)—, wherein R⁶, R⁷ and R⁸ are as defined above; and
R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different;
or a pharmaceutically acceptable salt, prodrug or active metabolite of a compound of the Formula I.

2. The method of claim 1, wherein the heterocyclic triazine compound of Formula I is a benzotriazine compound of the Formula I(a) 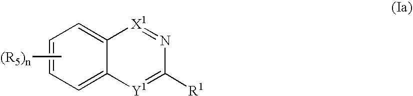
wherein
both X¹ and Y¹ are N-oxide; or
one of X¹ and Y¹ is N and the other is N or N-oxide;
each R⁵, which are the same or different are as defined for R² and R³; and
n is an integer from 1 to 4; or
a pharmaceutically acceptable salt, prodrug or active metabolite of a compound of the Formula I(a).

3. The method of claim 1, wherein said heterocyclic triazine compound is selected from the group consisting of:
Benzo[1,2,4]triazin-3-ylamine, Benzo[1,2,4]triazin-3-yl-pyridin-3-yl-amine, Benzo[1,2,4]triazin-3-yl-(4-piperazin-1-yl-phenyl)-amine, 7-(1H-Pyrazol-4-yl)-benzo[1,2,4]triazin-3-ylamine, 1-Benzo[1,2,4]triazin-3-yl-3-[5-chloro-2-(3-dimethylamino-propoxy)-phenyl]-urea, Benzo[1,2,4]triazin-3-yl-(5-methyl-1H-pyrazol-3-yl)-amine, 3-(1H-Pyrazol-4-yl)-benzo[1,2,4]triazine, 7-(2H-Pyrazol-3-yl)-benzo[1,2,4]triazin-3-ylamine, 3-(1H-Pyrazol-3-yl)-benzo[1,2,4]triazine, 1-Benzo[1,2,4]triazin-6-yl-3-pyrazin-2-yl-urea, Benzo[1,2,4]triazin-3-yl-(6-piperazin-1-yl-pyridin-3-yl)-amine, (6-Chloro-benzo[1,2,4]triazin-3-yl)-pyridin-3-yl-amine, (6-Phenyl-benzo[1,2,4]triazin-3-yl)-pyridin-3-yl-amine, 6-Piperazin-1-yl-benzo[1,2,4]triazin-3-ylamine, 6-(Piperidin-4-ylmethoxy)-benzo[1,2,4]triazin-3-ylamine, 6-[2-(3,4-Dichloro-phenyl)-ethoxy]-benzo[1,2,4]triazin-3-ylamine, Benzo[1,2,4]triazin-3-yl-quinolin-3-yl-amine, 4-(Benzo[1,2,4]triazin-3-ylamino)-benzonitrile, 4-Amino-N-[4-(benzo[1,2,4]triazin-3-ylamino)-phenyl]-2-(3,4-dichloro-phenyl)-butyramide, Benzo[1,2,4]triazin-3-yl-[4-([1,4]diazepane-1-sulfonyl)-phenyl]-amine, 1-[2-(2-Amino-ethoxy)-5-chloro-phenyl]-3-[1,2,4]triazin-3-yl-urea, 6-(2-Amino-ethoxy)-benzo[1,2,4]triazin-3-ylamine, 6-(2-Methylamino-ethoxy)-benzo[1,2,4]triazin-3-ylamine, 6-(2-Dimethylamino-ethoxy)-benzo[1,2,4]triazin-3-ylamine, [6-(2-Amino-ethoxy)-benzo[1,2,4]triazin-3-yl]-pyridin-3-yl-amine, [6-(2-Methylamino-ethoxy)-benzo[1,2,4]triazin-3-yl]-pyridin-3-yl-amine, [6-(2-Dimethylamino-ethoxy)-benzo[1,2,4]triazin-3-yl]-pyridin-3-yl-amine, Benzo[1,2,4]triazin-3-yl-(5-piperazin-1-yl-pyridin-2-yl)-amine, 1-[5-(3-Amino-propoxy)-2-chloro-phenyl]-3-benzo[1,2,4]triazin-3-yl-urea, 1-(3-Amino-benzo[1,2,4]triazin-7-yl)-3-pyrazin-2-yl-urea, Pyrido[4,3-e][1,2,4]triazin-3-ylamine, N*1*-Pyrido[4,3-e][1,2,4]triazin-3-yl-ethane-1,2-diamine, N*1*-Pyrido[4,3-e][1,2,4]triazin-3-yl-ethane-1,2-diamine, 7-Pyridin-4-yl-benzo[1,2,4]triazin-3-ylamine, 7-(1H-Pyrrolo[2,3-b]pyridin-4-yl)-benzo[1,2,4]triazin-3-ylamine, 3-(Pyridin-3-ylamino)-benzo[1,2,4]triazine-7-carboxylic acid (1-benzyl-pyrrolidin-3-yl)-amide, 1-[3-(1-Benzyl-pyrrolidin-3-ylamino)-benzo[1,2,4]triazin-6-yl]-3-pyrazin-2-yl-urea, Benzo[1,2,4]triazin-3-yl-(1H-pyrazol-3-yl)-amine, 4-Amino-N-(3-amino-benzo[1,2,4]triazin-6-yl)-2-(3,4-dichloro-phenyl)-butyramide, 1-[2-(3-Amino-propoxy)-5-chloro-phenyl]-3-(6-chloro-benzo[1,2,4]triazin-3-yl)-urea, 1-[2-(3-Amino-propoxy)-5-chloro-phenyl]-3-(7-fluoro-benzo[1,2,4]triazin-3-yl)-urea, N-[3-(Benzo[1,2,4]triazin-3-ylamino)-4-methyl-phenyl]-4-(4-methyl-piperazin-1-ylmethyl)-benzamide, 7-Fluoro-6-piperazin-1-yl-benzo[1,2,4]triazin-3-ylamine, (6-Phenyl-benzo[1,2,4]triazin-3-yl)-(3-piperazin-1-yl-phenyl)-amine, 3-Piperazin-1-yl-7-(1H-pyrazol-3-yl)-benzo[1,2,4]triazine, 1-[3-(4-Methyl-piperazin-1-yl)-benzo[1,2,4]triazin-7-yl]-3-pyrazin-2-yl-urea, (6-Phenyl-benzo[1,2,4]triazin-3-yl)-(4-piperazin-1-yl-phenyl)-amine, (6-Chloro-benzo[1,2,4]triazin-3-yl)-(4-piperazin-1-yl-phenyl)-amine.

4. The method of claim 1, wherein at least one of X¹ and Y¹ is N-oxide.

5. The method of claim 4, wherein the heterocyclic triazine compound is selected from the group consisting of:
1-Oxy-benzo[1,2,4]triazin-3-ylamine, (1-Oxy-benzo[1,2,4]triazin-3-yl)-pyridin-3-yl-amine, (1-Oxy-benzo[1,2,4]triazin-3-yl)-(4-piperazin-1-yl-phenyl)-amine, 1-Oxy-7-(1H-pyrazol-4-yl)-benzo[1,2,4]triazin-3-ylamine, (5-Methyl-1H-pyrazol-3-yl)-(1-oxy-benzo[1,2,4]triazin-3-yl)-amine, 3-(1H-Pyrazol-4-yl)-benzo[1,2,4]triazine 1-oxide, 1-Oxy-7-(2H-pyrazol-3-yl)-benzo[1,2,4]triazin-3-ylamine, 3-(1H-Pyrazol-3-yl)-benzo[1,2,4]triazine 1-oxide, (1-Oxy-benzo[1,2,4]triazin-3-yl)-(6-piperazin-1-yl-pyridin-3-yl)-amine, (6-Chloro-1-oxy-benzo[1,2,4]triazin-3-yl)-pyridin-3-yl-amine, (1-Oxy-6-phenyl-benzo[1,2,4]triazin-3-yl)-pyridin-3-yl-amine, 1-Oxy-6-piperazin-1-yl-benzo[1,2,4]triazin-3-ylamine, 1-Oxy-6-(piperidin-4-ylmethoxy)-benzo[1,2,4]triazin-3-ylamine.hydrochloride, 6-[2-(3,4-Dichloro-phenyl)-ethoxy]-1-oxy-benzo[1,2,4]triazin-3-ylamine, 1-(3-Amino-1-oxy-benzo[1,2,4]triazin-6-yl)-3-pyrazin-2-yl-urea, 4-(1-Oxy-benzo[1,2,4]triazin-3-ylamino)-benzonitrile, (1-Oxy-benzo[1,2,4]triazin-3-yl)-quinolin-3-yl-amine, 4-Amino-2-(3,4-dichloro-phenyl)-N-[4-(1-oxy-benzo[1,2,4]triazin-3-ylamino)-phenyl]-butyramide, [4-([1,4]Diazepane-1-sulfonyl)-phenyl]-(1-oxy-benzo[1,2,4]triazin-3-yl)-amine, 1-[2-(2-Amino-ethoxy)-5-chloro-phenyl]-3-(1-oxy-[1,2,4]triazin-3-yl)-urea, 6-(2-Amino-ethoxy)-1-oxy-benzo[1,2,4]triazin-3-ylamine, 6-(2-Methylamino-ethoxy)-1-oxy-benzo[1,2,4]triazin-3-ylamine, 6-(2-Dimethylamino-ethoxy)-1-oxy-benzo[1,2,4]triazin-3-ylamine, [6-(2-Amino-ethoxy)-1-oxy-benzo[1,2,4]triazin-3-yl]-pyridin-3-yl-amine, [6-(2-Methylamino-ethoxy)-1-oxy-benzo[1,2,4]triazin-3-yl]-pyridin-3-yl-amine, [6-(2-Dimethylamino-ethoxy)-1-oxy-benzo[1,2,4]triazin-3-yl]-pyridin-3-yl-amine, (1-Oxy-benzo[1,2,4]triazin-3-yl)-(5-piperazin-1-yl-pyridin-2-yl)-amine, 1-[5-(3-Amino-propoxy)-2-chloro-phenyl]-3-(1-oxy-benzo[1,2,4]triazin-3-yl)-urea, 1-(3-Amino-1-oxy-benzo[1,2,4]triazin-7-yl)-3-pyrazin-2-yl-urea, 1-Oxy-pyrido[4,3-e][1,2,4]triazin-3-ylamine, N*1*-(1-Oxy-pyrido[4,3-e][1,2,4]triazin-3-yl)-ethane-1,2-diamine, 1-Oxy-7-pyridin-4-yl-benzo[1,2,4]triazin-3-ylamine, 1-Oxy-7-(1H-pyrrolo[2,3-b]pyridin-4-yl)-benzo[1,2,4]triazin-3-ylamine, 1-Oxy-3-(pyridin-3-ylamino)-benzo[1,2,4]triazine-7-carboxylic acid (1-benzyl-pyrrolidin-3-yl)-amide, 1-[3-(1-Benzyl-pyrrolidin-3-ylamino)-1-oxy-benzo[1,2,4]triazin-6-yl]-3-pyrazin-2-yl-urea, (1-Oxy-benzo[1,2,4]triazin-3-yl)-(1H-pyrazol-3-yl)-amine, 4-Amino-N-(3-amino-1-oxy-benzo[1,2,4]triazin-6-yl)-2-(3,4-dichloro-phenyl)-butyramide, 1-[2-(3-Amino-propoxy)-5-chloro-phenyl]-3-(6-chloro-1-oxy-benzo[1,2,4]triazin-3-yl)-urea, 1-[2-(3-Amino-propoxy)-5-chloro-phenyl]-3-(7-fluoro-1-oxy-benzo[1,2,4]triazin-3-yl)-urea, N-[4-Methyl-3-(1-oxy-benzo[1,2,4]triazin-3-ylamino)-phenyl]-4-(4-methyl-piperazin-1-ylmethyl)-benzamide, 7-Fluoro-1-oxy-6-piperazin-1-yl-benzo[1,2,4]triazin-3-ylamine, (1-Oxy-6-phenyl-benzo[1,2,4]triazin-3-yl)-(3-piperazin-1-yl-phenyl)-amine, 3-Piperazin-1-yl-7-(1H-pyrazol-3-yl)-benzo[1,2,4]triazine 1-oxide, 1-[3-(4-Methyl-piperazin-1-yl)-1-oxy-benzo[1,2,4]triazin-7-yl]-3-pyrazin-2-yl-urea, (1-Oxy-6-phenyl-benzo[1,2,4]triazin-3-yl)-(4-piperazin-1-yl-phenyl)-amine, (6-Chloro-1-oxy-benzo[1,2,4]triazin-3-yl)-(4-piperazin-1-yl-phenyl)-amine.

6. The method of claim 4, wherein the heterocyclic triazine compound is selected from the group consisting of:
(1,4-Dioxy-benzo[1,2,4]triazin-3-yl)-pyridin-3-yl-amine, 4-(1,4-Dioxy-benzo[1,2,4]triazin-3-ylamino)-benzonitrile, (1,4-Dioxy-benzo[1,2,4]triazin-3-yl)-quinolin-3-yl-amine, 4-Amino-2-(3,4-dichloro-phenyl)-N-[4-(1,4-dioxy-benzo[1,2,4]triazin-3-ylamino)-phenyl]-butyramide, [4-([1,4]Diazepane-1-sulfonyl)-phenyl]-(1,4-dioxy-benzo[1,2,4]triazin-3-yl)-amine, 1-[2-(2-Amino-ethoxy)-5-chloro-phenyl]-3-(1,4-dioxy-[1,2,4]triazin-3-yl)-urea, 3-Amino-6-(2-amino-ethoxy)-1-hydroxy-4-oxy-benzo[1,2,4]triazin-1-ium, 6-(2-Methylamino-ethoxy)-1,4-dioxy-benzo[1,2,4]triazin-3-ylamine, 6-(2-Dimethylamino-ethoxy)-1,4-dioxy-benzo[1,2,4]triazin-3-ylamine, 6-(2-Amino-ethoxy)-1-hydroxy-4-oxy-3-(pyridin-3-ylamino)-benzo[1,2,4]triazin-1-ium, 1-Hydroxy-6-(2-methylamino-ethoxy)-4-oxy-3-(pyridin-3-ylamino)-benzo[1,2,4]triazin-1-ium, 6-(2-Dimethylamino-ethoxy)-1-hydroxy-4-oxy-3-(pyridin-3-ylamino)-benzo[1,2,4]triazin-1-ium, (1,4-Dioxy-benzo[1,2,4]triazin-3-yl)-(5-piperazin-1-yl-pyridin-2-yl)-amine, 1-[5-(3-Amino-propoxy)-2-chloro-phenyl]-3-(1,4-dioxy-benzo[1,2,4]triazin-3-yl)-urea, 1-(3-Amino-1,4-dioxy-benzo[1,2,4]triazin-7-yl)-3-pyrazin-2-yl-urea, 1-(3-Amino-1,4-dioxy-benzo[1,2,4]triazin-6-yl)-3-pyrazin-2-yl-urea, 1,4-Dioxy-pyrido[4,3-e][1,2,4]triazin-3-ylamine, N*1*-(1,4-Dioxy-pyrido[4,3-e][1,2,4]triazin-3-yl)-ethane-1,2-diamine, (1,4-Dioxy-benzo[1,2,4]triazin-3-yl)-(4-piperazin-1-yl-phenyl)-amine, 1,4-Dioxy-3-(pyridin-3-ylamino)-benzo[1,2,4]triazine-7-carboxylic acid (1-benzyl-pyrrolidin-3-yl)-amide, 1,4-Dioxy-7-(1H-pyrazol-4-yl)-benzo[1,2,4]triazin-3-ylamine, 1,4-Dioxy-7-pyridin-4-yl-benzo[1,2,4]triazin-3-ylamine, 1,4-Dioxy-7-(1H-pyrrolo[2,3-b]pyridin-4-yl)-benzo[1,2,4]triazin-3-ylamine, 1-[3-(1-Benzyl-pyrrolidin-3-ylamino)-1,4-dioxy-benzo[1,2,4]triazin-6-yl]-3-pyrazin-2-yl-urea, (1,4-Dioxy-benzo[1,2,4]triazin-3-yl)-(1H-pyrazol-3-yl)-amine, (1,4-Dioxy-benzo[1,2,4]triazin-3-yl)-(5-methyl-1H-pyrazol-3-yl)-amine, 1,4-Dioxy-7-(1H-pyrazol-4-yl)-benzo[1,2,4]triazin-3-ylamine, 1-[5-Chloro-2-(3-dimethylamino-propoxy)-phenyl]-3-(1,4-dioxy-benzo[1,2,4]triazin-3-yl)-urea, 3-(1H-Pyrazol-4-yl)-benzo[1,2,4]triazine 1,4-dioxide, 1,4-Dioxy-7-(2H-pyrazol-3-yl)-benzo[1,2,4]triazin-3-ylamine, 3-(1H-Pyrazol-3-yl)-benzo[1,2,4]triazine 1,4-dioxide, 1-(1,4-Dioxy-benzo[1,2,4]triazin-6-yl)-3-pyrazin-2-yl-urea, (1,4-Dioxy-benzo[1,2,4]triazin-3-yl)-(6-piperazin-1-yl-pyridin-3-yl)-amine, (6-Chloro-1,4-dioxy-benzo[1,2,4]triazin-3-yl)-pyridin-3-yl-amine, (1,4-Dioxy-6-phenyl-benzo[1,2,4]triazin-3-yl)-pyridin-3-yl-amine, 1,4-Dioxy-6-piperazin-1-yl-benzo[1,2,4]triazin-3-ylamine, 7-Fluoro-1,4-dioxy-6-piperazin-1-yl-benzo[1,2,4]triazin-3-ylamine, 3-Piperazin-1-yl-7-(1H-pyrazol-3-yl)-benzo[1,2,4]triazin 1,4-dioxide, (1,4-Dioxy-6-phenyl-benzo[1,2,4]triazin-3-yl)-(3-piperazin-1-yl-phenyl)-amine, 1-[3-(4-Methyl-piperazin-1-yl)-1,4-dioxy-benzo[1,2,4]triazin-7-yl]-3-pyrazin-2-yl-urea, 4-Amino-N-(3-amino-1,4-dioxy-benzo[1,2,4]triazin-6-yl)-2-(3,4-dichloro-phenyl)-butyramide, 1,4-Dioxy-6-(piperidin-4-ylmethoxy)-benzo[1,2,4]triazin-3-ylamine, 6-[2-(3,4-Dichloro-phenyl)-ethoxy]-1,4-dioxy-benzo[1,2,4]triazin-3-ylamine, 1-[2-(3-Amino-propoxy)-5-chloro-phenyl]-3-(6-chloro-1,4-dioxy-benzo[1,2,4]triazin-3-yl)-urea, 1,4-Dioxy-7-(1H-pyrazol-4-yl)-benzo[1,2,4]triazin-3-ylamine, 1-[2-(3-Amino-propoxy)-5-chloro-phenyl]-3-(7-fluoro-1,4-dioxy-benzo[1,2,4]triazin-3-yl)-urea, N-[3-(1,4-Dioxy-benzo[1,2,4]triazin-3-ylamino)-4-methyl-phenyl]-4-(4-methyl-piperazin-1-ylmethyl)-benzamide, (6-Chloro-1,4-dioxy-benzo[1,2,4]triazin-3-yl)-(4-piperazin-1-yl-phenyl)-amine, (1,4-Dioxy-6-phenyl-benzo[1,2,4]triazin-3-yl)-(4-piperazin-1-yl-phenyl)-amine.

7. The method of claim 4, wherein said heterocyclic triazine compound is a prodrug selectively reduced to a therapeutically active metabolite in a hypoxic environment.

8. The method of claim 7, wherein said prodrug compound has a one electron reduction potential in the range of from about −300 mV to about −550 mV.

9. The method of claim 8, wherein said prodrug compound has a one electron reduction potential in the range of from about −400 mV to about −510 mV.

10. A method for inhibiting the proliferation of cancer cells in a mammal, comprising administering to said mammal a therapeutically effective amount of a protein kinase inhibitor prodrug of the Formula I: 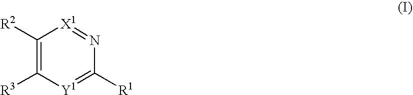
Wherein:
both X¹ and Y¹ are N-oxide; or
one of X¹ and Y¹ is N and the other is N-oxide;
R¹ is H, OH, NH₂, NHR⁴, a 5- to 7-membered heterocyclic ring which is unsaturated and which may contain one or more heteroatoms selected from O, N and S and which is unsubstituted or substituted on any ring carbon or ring heteroatom, or an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system;
R² and R³ are each independently selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸), —N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸)
wherein A is a C₁-C₆ alkylidine group that is optionally interrupted by —O—, —S—, —C(O)— or —N(R⁶), and wherein
R² and R³ may form, together with the carbon atoms to which they are attached, a fused benzene ring or a fused 5- to 7-membered heterocyclic ring which is saturated or unsaturated and which may contain one or more heteroatoms selected from O, N and S, the benzene ring or heterocyclic ring being unsubstituted or substituted;
wherein R⁶ is H, C₁-C₆ alkyl which is unsubstituted or substituted, C₃-C₁₀ cycloalkyl which is unsubstituted or substituted or a 5- to 7-membered heterocyclic ring which is unsaturated or saturated and which contains one or more heteroatoms selected from O, N and S, and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different; and wherein
R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;
p is 0 or an integer from 1 to 5;
q is an integer from 1 to 6;
R⁴ is H, C₁-C₆ alkyl which is unsubstituted or substituted, C₃-C₁₀ cycloalkyl which is unsubstituted or substituted, aryl which is unsubstituted or substituted, a 5- to 7-membered heterocyclic group which is unsaturated or saturated, which contains one or more heteroatoms selected from O, N and S and which is unsubstituted or substituted on any ring carbon or ring heteroatom, the carbocyclic group or heterocyclic group R⁴ being optionally substituted by one or more substituent selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸)—N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸) wherein A is a C₁-C₆ alkylidine group that is optionally interrupted by —O—, —S—, —C(O)— or —N(R⁶)—, wherein R⁶, R⁷ and R⁸ are as defined above; and
R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different
or a pharmaceutically acceptable salt of a compound of the Formula I.

11. The method of claim 10, wherein the prodrug is a compound of the Formula I(a) 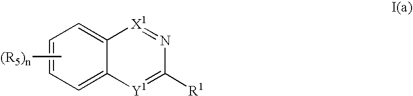
wherein
both X¹ and Y¹ are N-oxide; or
one of X¹ and Y¹ is N and the other is N-oxide;
each R⁵, which are the same or different are as defined for R² and R³;
n is an integer from 1 to 4; or
a pharmaceutically acceptable salt of a compound of the Formula I(a).

12. The method of claim 10, wherein said prodrug is selectively reduced to a therapeutically active metabolite via reduction in a hypoxic environment.

13. The method of claim 12, wherein said prodrug has a one electron reduction potential in the range of from about −300 mV to about −550 mV.

14. The method of claim 13, wherein said prodrug has a one electron reduction potential in the range of from about −400 mV to about −510 mV.

15. The method of claim 10, further comprising the administration of ionizing radiation to said mammal.

16. The method of claim 10, further comprising the administration of a chemotherapeutic agent that imparts oxidative damage to DNA of said cancer cells.

17. The method of claim 16, wherein said chemotherapeutic agent is Tirapazamine.

18. The method of claim 15, wherein said prodrug administration enhances the cytotoxicity of said ionizing radiation in hypoxic tumor cells.

19. The method of claim 17, wherein said prodrug administration enhances the cytotoxicity of said Tirapazamine in hypoxic tumor cells.

20. The method of claim 10, wherein R¹ is H, OH, NHR⁴, a 5-to 7-membered heterocyclic ring which is unsaturated and may contain one or more heteroatoms selected from O, N and S, and which is unsubstituted or substituted on any ring carbon or ring heteroatom, or an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system.

21. A method of inhibiting the activity of a protein kinase comprising exposing said kinase to an effective amount of a kinase inhibitor of the Formula I: 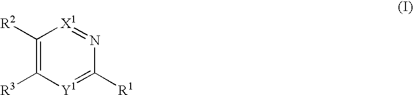
Wherein:
one of X¹ and Y¹ is N and the other is N-oxide;
R¹ is H, OH, NH₂, NHR⁴, a 5- to 7-membered heterocyclic ring which is unsaturated and which may contain one or more heteroatoms selected from O, N and S and which is unsubstituted or substituted on any ring carbon or ring heteroatom, or an aromatic or heteroaromatic ring optionally substituted by halogen, hydroxyl, —OR¹⁰, —SR¹⁰, —SO₂R¹⁰, SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system;
R² and R³ are each independently selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸), —N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸)
wherein A is a C₁-C₆ alkylidine group that is optionally interrupted by —O—, —S—, —C(O)— or —N(R⁶), and wherein
R² and R³ may form, together with the carbon atoms to which they are attached, a fused benzene ring or a fused 5- to 7-membered heterocyclic ring which is saturated or unsaturated and which may contain one or more heteroatoms selected from O, N and S, the benzene ring or heterocyclic ring being unsubstituted or substituted;
wherein R⁶ is H, C₁-C₆ alkyl which is unsubstituted or substituted, C₃-C₁₀ cycloalkyl which is unsubstituted or substituted or a 5- to 7-membered heterocyclic ring which is unsaturated or saturated and which contains one or more heteroatoms selected from O, N and S, and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system, and wherein more than one R⁶ attached to the same nitrogen atom is the same or different; and wherein
R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;
p is 0 or an integer from 1 to 5;
q is an integer from 1 to 6;
R⁴ is H, C₁-C₆ alkyl which is unsubstituted or substituted, C₃-C₁₀ cycloalkyl which is unsubstituted or substituted, aryl which is unsubstituted or substituted, a 5- to 7-membered heterocyclic group which is unsaturated or saturated, which contains one or more heteroatoms selected from O, N and S and which is unsubstituted or substituted on any ring carbon or ring heteroatom, the carbocyclic group or heterocyclic group R⁴ being optionally substituted by one or more substituent selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸)—N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚ(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸) wherein A is a C₁-C₆ alkylidine group that is optionally interrupted by —O—, —S—, —C(O)— or —N(R⁶)—, wherein R⁶, R⁷ and R⁸ are as defined above; and
R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different.

22. The method of claim 21, wherein said kinase inhibitor is a benzotriazine compound of the formula I(a) 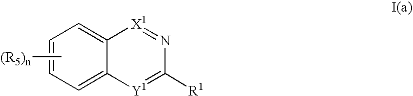
wherein
one of X¹ and Y¹ is N and the other is N or N-oxide;
each R⁵, which are the same or different are as defined for R² and R³;
n is an integer from 1 to 4; or

23. The method of claim 21, wherein said kinase inhibitor is selected from the group consisting of:
Benzo[1,2,4]triazin-3-ylamine, Benzo[1,2,4]triazin-3-yl-pyridin-3-yl-amine, Benzo[1,2,4]triazin-3-yl-(4-piperazin-1-yl-phenyl)-amine, 7-(1H-Pyrazol-4-yl)-benzo[1,2,4]triazin-3-ylamine, 1-Benzo[1,2,4]triazin-3-yl-3-[5-chloro-2-(3-dimethylamino-propoxy)-phenyl]-urea, Benzo[1,2,4]triazin-3-yl-(5-methyl-1H-pyrazol-3-yl)-amine, 3-(1H-Pyrazol-4-yl)-benzo[1,2,4]triazine, 7-(2H-Pyrazol-3-yl)-benzo[1,2,4]triazin-3-ylamine, 3-(1H-Pyrazol-3-yl)-benzo[1,2,4]triazine, 1-Benzo[1,2,4]triazin-6-yl-3-pyrazin-2-yl-urea, Benzo[1,2,4]triazin-3-yl-(6-piperazin-1-yl-pyridin-3-yl)-amine, (6-Chloro-benzo[1,2,4]triazin-3-yl)-pyridin-3-yl-amine, (6-Phenyl-benzo[1,2,4]triazin-3-yl)-pyridin-3-yl-amine, 6-Piperazin-1-yl-benzo[1,2,4]triazin-3-ylamine, 6-(Piperidin-4-ylmethoxy)-benzo[1,2,4]triazin-3-ylamine, 6-[2-(3,4-Dichloro-phenyl)-ethoxy]-benzo[1,2,4]triazin-3-ylamine, Benzo[1,2,4]triazin-3-yl-quinolin-3-yl-amine, 4-(Benzo[1,2,4]triazin-3-ylamino)-benzonitrile, 4-Amino-N-[4-(benzo[1,2,4]triazin-3-ylamino)-phenyl]-2-(3,4-dichloro-phenyl)-butyramide, Benzo[1,2,4]triazin-3-yl-[4-([1,4]diazepane-1-sulfonyl)-phenyl]-amine, 1-[2-(2-Amino-ethoxy)-5-chloro-phenyl]-3-[1,2,4]triazin-3-yl-urea, 6-(2-Amino-ethoxy)-benzo[1,2,4]triazin-3-ylamine, 6-(2-Methylamino-ethoxy)-benzo[1,2,4]triazin-3-ylamine, 6-(2-Dimethylamino-ethoxy)-benzo[1,2,4]triazin-3-ylamine, [6-(2-Amino-ethoxy)-benzo[1,2,4]triazin-3-yl]-pyridin-3-yl-amine, [6-(2-Methylamino-ethoxy)-benzo[1,2,4]triazin-3-yl]-pyridin-3-yl-amine, [6-(2-Dimethylamino-ethoxy)-benzo[1,2,4]triazin-3-yl]-pyridin-3-yl-amine, Benzo[1,2,4]triazin-3-yl-(5-piperazin-1-yl-pyridin-2-yl)-amine, 1-[5-(3-Amino-propoxy)-2-chloro-phenyl]-3-benzo[1,2,4]triazin-3-yl-urea, 1-(3-Amino-benzo[1,2,4]triazin-7-yl)-3-pyrazin-2-yl-urea, Pyrido[4,3-e][1,2,4]triazin-3-ylamine, N*1*-Pyrido[4,3-e][1,2,4]triazin-3-yl-ethane-1,2-diamine, N*1*-Pyrido[4,3-e][1,2,4]triazin-3-yl-ethane-1,2-diamine, 7-Pyridin-4-yl-benzo[1,2,4]triazin-3-ylamine, 7-(1H-Pyrrolo[2,3-b]pyridin-4-yl)-benzo[1,2,4]triazin-3-ylamine, 3-(Pyridin-3-ylamino)-benzo[1,2,4]triazine-7-carboxylic acid (1-benzyl-pyrrolidin-3-yl)-amide, 1-[3-(1-Benzyl-pyrrolidin-3-ylamino)-benzo[1,2,4]triazin-6-yl]-3-pyrazin-2-yl-urea, Benzo[1,2,4]triazin-3-yl-(1H-pyrazol-3-yl)-amine, 4-Amino-N-(3-amino-benzo[1,2,4]triazin-6-yl)-2-(3,4-dichloro-phenyl)-butyramide, 1-[2-(3-Amino-propoxy)-5-chloro-phenyl]-3-(6-chloro-benzo[1,2,4]triazin-3-yl)-urea, 1-[2-(3-Amino-propoxy)-5-chloro-phenyl]-3-(7-fluoro-benzo[1,2,4]triazin-3-yl)-urea, N-[3-(Benzo[1,2,4]triazin-3-ylamino)-4-methyl-phenyl]-4-(4-methyl-piperazin-1-ylmethyl)-benzamide, 7-Fluoro-6-piperazin-1-yl-benzo[1,2,4]triazin-3-ylamine, (6-Phenyl-benzo[1,2,4]triazin-3-yl)-(3-piperazin-1-yl-phenyl)-amine, 3-Piperazin-1-yl-7-(1H-pyrazol-3-yl)-benzo[1,2,4]triazine, 1-[3-(4-Methyl-piperazin-1-yl)-benzo[1,2,4]triazin-7-yl]-3-pyrazin-2-yl-urea, (6-Phenyl-benzo[1,2,4]triazin-3-yl)-(4-piperazin-1-yl-phenyl)-amine, (6-Chloro-benzo[1,2,4]triazin-3-yl)-(4-piperazin-1-yl-phenyl)-amine.

24. The method of claim 21, wherein said kinase inhibitor is selected from the group consisting of:
1-Oxy-benzo[1,2,4]triazin-3-ylamine, (1-Oxy-benzo[1,2,4]triazin-3-yl)-pyridin-3-yl-amine, (1-Oxy-benzo[1,2,4]triazin-3-yl)-(4-piperazin-1-yl-phenyl)-amine, 1-Oxy-7-(1H-pyrazol-4-yl)-benzo[1,2,4]triazin-3-ylamine, (5-Methyl-1H-pyrazol-3-yl)-(1-oxy-benzo[1,2,4]triazin-3-yl)-amine, 3-(1H-Pyrazol-4-yl)-benzo[1,2,4]triazine 1-oxide, 1-Oxy-7-(2H-pyrazol-3-yl)-benzo[1,2,4]triazin-3-ylamine, 3-(1H-Pyrazol-3-yl)-benzo[1,2,4]triazine 1-oxide, (1-Oxy-benzo[1,2,4]triazin-3-yl)-(6-piperazin-1-yl-pyridin-3-yl)-amine, (6-Chloro-1-oxy-benzo[1,2,4]triazin-3-yl)-pyridin-3-yl-amine, (1-Oxy-6-phenyl-benzo[1,2,4]triazin-3-yl)-pyridin-3-yl-amine, 1-Oxy-6-piperazin-1-yl-benzo[1,2,4]triazin-3-ylamine, 1-Oxy-6-(piperidin-4-ylmethoxy)-benzo[1,2,4]triazin-3-ylamine.hydrochloride, 6-[2-(3,4-Dichloro-phenyl)-ethoxy]-1-oxy-benzo[1,2,4]triazin-3-ylamine, 1-(3-Amino-l-oxy-benzo[1,2,4]triazin-6-yl)-3-pyrazin-2-yl-urea, 4-(1-Oxy-benzo[1,2,4]triazin-3-ylamino)-benzonitrile, (1-Oxy-benzo[1,2,4]triazin-3-yl)-quinolin-3-yl-amine, 4-Amino-2-(3,4-dichloro-phenyl)-N-[4-(1-oxy-benzo[1,2,4]triazin-3-ylamino)-phenyl]-butyramide, [4-([1,4]Diazepane-1-sulfonyl)-phenyl]-(1-oxy-benzo[1,2,4]triazin-3-yl)-amine, 1-[2-(2-Amino-ethoxy)-5-chloro-phenyl]-3-(1-oxy-[1,2,4]triazin-3-yl)-urea, 6-(2-Amino-ethoxy)-1-oxy-benzo[1,2,4]triazin-3-ylamine, 6-(2-Methylamino-ethoxy)-1-oxy-benzo[1,2,4]triazin-3-ylamine, 6-(2-Dimethylamino-ethoxy)-1-oxy-benzo[1,2,4]triazin-3-ylamine, [6-(2-Amino-ethoxy)-1-oxy-benzo[1,2,4]triazin-3-yl]-pyridin-3-yl-amine, [6-(2-Methylamino-ethoxy)-1-oxy-benzo[1,2,4]triazin-3-yl]-pyridin-3-yl-amine, [6-(2-Dimethylamino-ethoxy)-1-oxy-benzo[1,2,4]triazin-3-yl]-pyridin-3-yl-amine, (1-Oxy-benzo[1,2,4]triazin-3-yl)-(5-piperazin-1-yl-pyridin-2-yl)-amine, 1-[5-(3-Amino-propoxy)-2-chloro-phenyl]-3-(1-oxy-benzo[1,2,4]triazin-3-yl)-urea, 1-(3-Amino-1-oxy-benzo[1,2,4]triazin-7-yl)-3-pyrazin-2-yl-urea, 1-Oxy-pyrido[4,3-e][1,2,4]triazin-3-ylamine, N*1*-(1-Oxy-pyrido[4,3-e][1,2,4]triazin-3-yl)-ethane-1,2-diamine, 1-Oxy-7-pyridin-4-yl-benzo[1,2,4]triazin-3-ylamine, 1-Oxy-7-(1H-pyrrolo[2,3-b]pyridin-4-yl)-benzo[1,2,4]triazin-3-ylamine, 1-Oxy-3-(pyridin-3-ylamino)-benzo[1,2,4]triazine-7-carboxylic acid (1-benzyl-pyrrolidin-3-yl)-amide, 1-[3-(1-Benzyl-pyrrolidin-3-ylamino)-1-oxy-benzo[1,2,4]triazin-6-yl]-3-pyrazin-2-yl-urea, (1-Oxy-benzo[1,2,4]triazin-3-yl)-(1H-pyrazol-3-yl)-amine, 4-Amino-N-(3-amino-1-oxy-benzo[1,2,4]triazin-6-yl)-2-(3,4-dichloro-phenyl)-butyramide, 1-[2-(3-Amino-propoxy)-5-chloro-phenyl]-3-(6-chloro-1-oxy-benzo[1,2,4]triazin-3-yl)-urea, 1-[2-(3-Amino-propoxy)-5-chloro-phenyl]-3-(7-fluoro-1-oxy-benzo[1,2,4]triazin-3-yl)-urea, N-[4-Methyl-3-(1-oxy-benzo[1,2,4]triazin-3-ylamino)-phenyl]-4-(4-methyl-piperazin-1-ylmethyl)-benzamide, 7-Fluoro-1-oxy-6-piperazin-1-yl-benzo[1,2,4]triazin-3-ylamine, (1-Oxy-6-phenyl-benzo[1,2,4]triazin-3-yl)-(3-piperazin-1-yl-phenyl)-amine, 3-Piperazin-1-yl-7-(1H-pyrazol-3-yl)-benzo[1,2,4]triazine 1-oxide, 1-[3-(4-Methyl-piperazin-1-yl)-1-oxy-benzo[1,2,4]triazin-7-yl]-3-pyrazin-2-yl-urea, (1-Oxy-6-phenyl-benzo[1,2,4]triazin-3-yl)-(4-piperazin-1-yl-phenyl)-amine, (6-Chloro-1-oxy-benzo[1,2,4]triazin-3-yl)-(4-piperazin-1-yl-phenyl)-amine.

25. The method of claim 21, wherein said kinase inhibitor is generated in vivo by the reduction of a N-oxide prodrug thereof.

26. The method of claim 21, wherein said kinase is selected from the group consisting of: Arg, Abl, Aurora A, CDK1/cyclinB, CDK2/cyclinE, CHK1, c-RAF, cSRC, EGFR, ErbB4, GFR1, JNK1α1, KDR, MAPK2, MEK1, p70S6K, PDGFRβ, PKC θ, and Plk3, Flt-1, C=Kit, FGFR1, ERBBZ, CMet, TIEZ, RET, VEGFR, IGF-1R, Akt, PKA, P13K, PDK1, PDK2, Cdk2, Cdk4, Ck2, Myt1, Wee1, Auroa B, Plk, Bulb1, Bulb3, Chk2, ATM, ATR, and DNA-PK.

27. The method of claim 26, wherein said kinase is selected from the group consisting of: CK2, Arg, Abl, Aurora A, CDK1/Cyclin B, KDR, and P70S6.

28. A prodrug for the selective inhibition of hypoxic cancer cell proliferation having the Formula I: 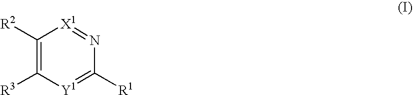
Wherein:
both X¹ and Y¹ are N-oxide; or
one of X¹ and Y¹ is N and the other is N-oxide;
R¹ is H, OH, NH₂, NHR⁴, a 5- to 7-membered heterocyclic ring which is unsaturated and which may contain one or more heteroatoms selected from O, N and S and which is unsubstituted or substituted on any ring carbon or ring heteroatom, or an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system;
R² and R³ are each independently selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸), —N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸)
wherein A is a C₁-C₆ alkylidine group that is optionally interrupted by —O—, —S—, —C(O)— or —N(R⁶), and wherein
R² and R³ may form, together with the carbon atoms to which they are attached, a fused benzene ring or a fused 5- to 7-membered heterocyclic ring which is saturated or unsaturated and which may contain one or more heteroatoms selected from O, N and S, the benzene ring or heterocyclic ring being unsubstituted or substituted;
wherein R⁶ is H, C₁-C₆ alkyl which is unsubstituted or substituted, C₃-C₁₀ cycloalkyl which is unsubstituted or substituted or a 5- to 7-membered heterocyclic ring which is unsaturated or saturated and which contains one or more heteroatoms selected from O, N and S, and which is unsubstituted or substituted on any ring carbon or ring heteroatom, an aromatic or heteroaromatic ring optionally substituted by halogen, hydroxyl, —OR¹⁰, —SR¹⁰, —SO₂R¹⁰, —SO₂N(R¹⁰)₂, —N(R¹⁰)₂, —N(R⁷)(R⁸), cyano, nitro, —COOR¹⁰, —C(O)N(R¹⁰)₂, —N(R¹⁰)C(O)R¹⁰, —N(R¹⁰)COOR¹⁰, —N(R¹⁰)CON(R¹⁰)₂, —N(R¹⁰)SO(R¹⁰), —N(R¹⁰)SO₂(R¹⁰), —C(O)R¹⁰ and aromatic or heteroaromatic ring optionally substituted by two R¹⁰ that may be taken together to form a fused bicyclic system and wherein more than one R⁶ attached to the same nitrogen atom is the same or different; and wherein
R⁷ and R⁸ form, together with the N atom to which they are attached, a 3- to 9-membered N-containing heterocyclic ring which is unsaturated or saturated and which may contain one or more heteroatoms selected from O, N and S and which is unsubstituted or substituted on any ring carbon or ring heteroatom;
p is 0 or an integer from 1 to 5;
q is an integer from 1 to 6;
R⁴ is H, C₁-C₆ alkyl which is unsubstituted or substituted, C₃-C₁₀ cycloalkyl which is unsubstituted or substituted, aryl which is unsubstituted or substituted, a 5- to 7-membered heterocyclic group which is unsaturated or saturated, which contains one or more heteroatoms selected from O, N and S and which is unsubstituted or substituted on any ring carbon or ring heteroatom, the carbocyclic group or heterocyclic group R⁴ being optionally substituted by one or more substituent selected from hydrogen, C₁-C₆ alkyl which is unsubstituted or substituted, C₃-C₁₀ cycloalkyl which is unsubstituted or substituted, aryl which is unsubstituted or substituted, a 5- to 7-membered heterocyclic ring which is saturated or unsaturated and which may contain one or more heteroatoms selected from O, N and S and which is unsubstituted or substituted on any ring carbon or ring heteroatom, C₁-C₆ alkoxy which is unsubstituted or substituted, C₃-C₁₀ cycloalkoxy which is unsubstituted or substituted, halogen, hydroxyl, —OR⁶, —SR⁶, —SO₂R⁶, —SO₂N(R⁶)₂, —SO₂N(R⁷)(R⁸), —N(R⁶)₂, —N(R⁷)(R⁸), cyano, nitro, —COOR⁶, —C(O)N(R⁶)₂, —C(O)N(R⁷)(R⁸), —N(R⁶)C(O)R⁶, —N(R⁶)COOR⁶, —N(R⁶)CON(R⁶)₂, —N(R⁶)CON(R⁷)(R⁸)—N(R⁶)SO(R⁶), —N(R⁶)SO₂(R⁶), —C(O)R⁶, OCH₂(CH₂)ₚN(R⁶)₂, —OCH₂(CH₂)ₚN(R⁷)(R⁸), —CH₂(CH₂)ₚN(R⁶)₂, —CH₂(CH₂)ₚN(R⁷)(R⁸), —C(O)NHCH₂(CH₂)ₚN(R⁶)₂, —C(O)NHCH₂(CH₂)ₚN(R⁷)(R⁸), —NH(CH₂)ₚN(R⁶)₂, —NH(CH₂)ₚN(R⁷)(R⁸), —OCH₂CH₂OR⁶, —NHC(O)CH₂(CH₂)ₚN(R⁷)(R⁸), —NHC(O)CH₂(CH₂)ₚN(R⁶)₂, —(CH₂)_{q}C(O)R⁶, —O(CH₂)_{q}C(O)R⁶, —O(CH₂)_{q}(OCH₂CH₂)_{q}OR⁶, -A-N(R⁶)₂ or -A-N(R⁷)(R⁸) wherein A is a C₁-C₆ alkylidine group that is optionally interrupted by —O—, —S—, —C(O)— or —N(R⁶)—, wherein R⁶, R⁷ and R⁸ are as defined above; and
R¹⁰ is H or C₁-C₆ alkyl which is unsubstituted or substituted, C₃-C₁₀ cycloalkyl which is unsubstituted or substituted or a 5- to 7-membered heterocyclic ring which is unsaturated or saturated which contains one or more heteroatoms selected from O, N and S and which is unsubstituted or substituted on any ring carbon or ring heteroatom and wherein more than one R¹⁰ attached to the same nitrogen atom is the same or different,
or a pharmaceutically acceptable salt thereof.

29. The prodrug of claim 28, wherein said prodrug is a compound of the Formula I(a): 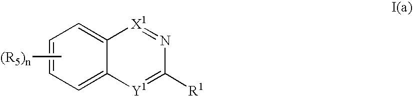
wherein
both X¹ and Y¹ are N-oxide; or
one of X¹ and Y¹ is N and the other is N-oxide;
each R⁵, which are the same or different are as defined for R² and R³;
n is an integer from 1 to 4; or
a pharmaceutically acceptable salt thereof.

30. The prodrug of claim 28, wherein said prodrug has a one electron reduction potential in the range of from about −400 mV to about −510 mV.

31. The prodrug of claim 28, wherein said prodrug undergoes reduction of at least one N-oxide moiety in a hypoxic environment to form a metabolite that inhibits protein kinase activity.

32. The prodrug of claim 31, wherein said metabolite inhibits the activity of protein kinases selected from the group consisting of: Arg, Abl, Aurora A, CDK1/cyclinB, CDK2/cyclinE, CHK1, c-RAF, cSRC, EGFR, ErbB4, GFR1, JNK1α1, KDR, MAPK2, MEK1, p70S6K, PDGFRβ, PKC θ, and Plk3, Flt-1, C=Kit, FGFR1, ERBBZ, CMet, TIEZ, RET, VEGFR, IGF-1R, Akt, PKA, P13K, PDK1, PDK2, Cdk2, Cdk4, Ck2, Myt1, Wee1, Auroa B, Plk, Bulb1, Bulb3, Chk2, ATM, ATR, and DNA-PK.

33. The prodrhg of claim 31, wherein said metabolite inhibits the activity of a proteinkinase selected from the group consisting of: CK2, Arg, Abl, Aurora A, CDK1/Cyclin B, KDR, and P70S6.

34. A pharmaceutical composition for treating a disease state associated with uncontrolled cellular proliferation comprising a pharmaceutically acceptable carrier, and a therapeutically effective amount of a protein kinase inhibitor prodrug of claim 28.

35. The pharmaceutical composition of claim 34, further comprising Tirapazamine.
